SEQUENCE LISTING (153 sequences)

SEQ ID NO: 1 - RNA, 20 nt, Artificial Sequence
  Guide sequence based on human DMD gene
agaucugucaaaucgccugc

SEQ ID NO: 2 - RNA, 20 nt, Artificial Sequence
  Guide sequence based on human DMD gene
auccauaugcuuuuaccugc

SEQ ID NO: 3 - RNA, 20 nt, Artificial Sequence
  Guide sequence based on human DMD gene
auucuuuuguucuucuagcc

SEQ ID NO: 4 - RNA, 20 nt, Artificial Sequence
  Guide sequence based on human DMD gene
aauuuuauucuucuuucucc

SEQ ID NO: 5 - RNA, 20 nt, Artificial Sequence
  Guide sequence based on human DMD gene
aucggccucccuaagcgcua

SEQ ID NO: 6 - RNA, 20 nt, Artificial Sequence
  Guide sequence based on human DMD gene
cucaagugauccgcccacau

SEQ ID NO: 7 - RNA, 20 nt, Artificial Sequence
  Guide sequence based on human DMD gene
aucuguccugguuaccuacc

SEQ ID NO: 8 - RNA, 20 nt, Artificial Sequence
  Guide sequence based on human DMD gene
cugaaguggugagacugauc

SEQ ID NO: 9 - RNA, 20 nt, Artificial Sequence
  Guide sequence based on human DMD gene
gugcacugguucagaacugc

SEQ ID NO: 10 - RNA, 20 nt, Artificial Sequence
  Guide sequence based on human DMD gene
aggcuucugaaccuucgccu

SEQ ID NO: 11 - RNA, 20 nt, Artificial Sequence
  Guide sequence based on human DMD gene
agcaaucuccccguauaguc

SEQ ID NO: 12 - RNA, 20 nt, Artificial Sequence
  Guide sequence based on human DMD gene
ugauguggccagacuauacg

SEQ ID NO: 13 - RNA, 20 nt, Artificial Sequence
  Guide sequence based on human DMD gene
agacuauacggggagauugc

SEQ ID NO: 14 - RNA, 20 nt, Artificial Sequence
  Guide sequence based on human DMD gene
guagaguuagaaucccuguu

SEQ ID NO: 15 - RNA, 20 nt, Artificial Sequence
  Guide sequence based on human DMD gene
ugacuaaaguuuagucaagc

SEQ ID NO: 16 - RNA, 20 nt, Artificial Sequence
  Guide sequence based on human DMD gene
cauucguauucugggugucu

SEQ ID NO: 17 - RNA, 20 nt, Artificial Sequence
  Guide sequence based on human DMD gene
aagagagauacggguuaauc

SEQ ID NO: 18 - RNA, 20 nt, Artificial Sequence
  Guide sequence based on human DMD gene
aucaaugucaggcaucaguc

SEQ ID NO: 19 - RNA, 20 nt, Artificial Sequence
  Guide sequence based on human DMD gene
gguacaaacacaguauacga

SEQ ID NO: 20 - RNA, 20 nt, Artificial Sequence
  Guide sequence based on human DMD gene
uacagucucuugcaauuuac

SEQ ID NO: 21 - RNA, 20 nt, Artificial Sequence
  Guide sequence based on human DMD gene
auuccgggugcugcauuuuc

SEQ ID NO: 22 - RNA, 20 nt, Artificial Sequence
  Guide sequence based on human DMD gene
cagccucaguguaauccauu

SEQ ID NO: 23 - RNA, 20 nt, Artificial Sequence
  Guide sequence based on human DMD gene
auaguauauuugaucuagcu

SEQ ID NO: 24 - RNA, 20 nt, Artificial Sequence
  Guide sequence based on human DMD gene
gauuguggucaagccaucuc

SEQ ID NO: 25 - RNA, 20 nt, Artificial Sequence
  Guide sequence based on human DMD gene
guaugcucuguacucagaau

SEQ ID NO: 26 - RNA, 20 nt, Artificial Sequence
  Guide sequence based on human DMD gene
auccuuguucugcuacuuac

SEQ ID NO: 27 - RNA, 20 nt, Artificial Sequence
  Guide sequence based on human DMD gene
uuaccaaauggauuacacug

SEQ ID NO: 28 - RNA, 20 nt, Artificial Sequence
  Guide sequence based on human DMD gene
uuuggagagcaucagauuac

SEQ ID NO: 29 - RNA, 20 nt, Artificial Sequence
  Guide sequence based on human DMD gene
uuuaaggaccacaaucucag

SEQ ID NO: 30 - RNA, 20 nt, Artificial Sequence
  Guide sequence based on human DMD gene
ucaucuucauuggccucaug

SEQ ID NO: 31 - RNA, 20 nt, Artificial Sequence
  Guide sequence based on human DMD gene
auaugauguucuaccacaug

SEQ ID NO: 32 - RNA, 20 nt, Artificial Sequence
  Guide sequence based on human DMD gene
gcugauuuucuaacgaauuu

SEQ ID NO: 33 - RNA, 20 nt, Artificial Sequence
  Guide sequence based on human DMD gene
gcugauuuucuaaugaauuu

SEQ ID NO: 34 - RNA, 20 nt, Artificial Sequence
  Guide sequence based on human DMD gene
guuuggugauucuuacggac

SEQ ID NO: 35 - RNA, 20 nt, Artificial Sequence
  Guide sequence based on human DMD gene
uauucauuaguucuggaggg

SEQ ID NO: 36 - RNA, 20 nt, Artificial Sequence
  Guide sequence based on human DMD gene
caacugcagcagcacgcauu

SEQ ID NO: 37 - RNA, 20 nt, Artificial Sequence
  Guide sequence based on human DMD gene
ugugccuucaauacauucca

SEQ ID NO: 38 - RNA, 20 nt, Artificial Sequence
  Guide sequence based on human DMD gene
uaaugugucuuguuucguaa

SEQ ID NO: 39 - RNA, 20 nt, Artificial Sequence
  Guide sequence based on human DMD gene
ucauauucuguaguacaagg

SEQ ID NO: 40 - RNA, 20 nt, Artificial Sequence
  Guide sequence based on human DMD gene
acaaaacuccauaauaguuc

SEQ ID NO: 41 - RNA, 20 nt, Artificial Sequence
  Guide sequence based on human DMD gene
uugcauagaguacguaacag

SEQ ID NO: 42 - RNA, 20 nt, Artificial Sequence
  Guide sequence based on human DMD gene
guugaaauuaaacuacacac

SEQ ID NO: 43 - RNA, 20 nt, Artificial Sequence
  Guide sequence based on human DMD gene
uguaugaugcuauaauacca

SEQ ID NO: 44 - DNA, 20 bp, Artificial Sequence
  Non-complementary strand of target sequence of human DMD gene
agatctgtcaaatcgcctgc

SEQ ID NO: 45 - DNA, 20 bp, Artificial Sequence
  Non-complementary strand of target sequence of human DMD gene
atccatatgcttttacctgc

SEQ ID NO: 46 - DNA, 20 bp, Artificial Sequence
  Non-complementary strand of target sequence of human DMD gene
attcttttgttcttctagcc

SEQ ID NO: 47 - DNA, 20 bp, Artificial Sequence
  Non-complementary strand of target sequence of human DMD gene
aattttattcttctttctcc

SEQ ID NO: 48 - DNA, 20 bp, Artificial Sequence
  Non-complementary strand of target sequence of human DMD gene
atcggcctccctaagcgcta

SEQ ID NO: 49 - DNA, 20 bp, Artificial Sequence
  Non-complementary strand of target sequence of human DMD gene
ctcaagtgatccgcccacat

SEQ ID NO: 50 - DNA, 20 bp, Artificial Sequence
  Non-complementary strand of target sequence of human DMD gene
atctgtcctggttacctacc

SEQ ID NO: 51 - DNA, 20 bp, Artificial Sequence
  Non-complementary strand of target sequence of human DMD gene
ctgaagtggtgagactgatc

SEQ ID NO: 52 - DNA, 20 bp, Artificial Sequence
  Non-complementary strand of target sequence of human DMD gene
gtgcactggttcagaactgc

SEQ ID NO: 53 - DNA, 20 bp, Artificial Sequence
  Non-complementary strand of target sequence of human DMD gene
aggcttctgaaccttcgcct

SEQ ID NO: 54 - DNA, 20 bp, Artificial Sequence
  Non-complementary strand of target sequence of human DMD gene
agcaatctccccgtatagtc

SEQ ID NO: 55 - DNA, 20 bp, Artificial Sequence
  Non-complementary strand of target sequence of human DMD gene
tgatgtggccagactatacg

SEQ ID NO: 56 - DNA, 20 bp, Artificial Sequence
  Non-complementary strand of target sequence of human DMD gene
agactatacggggagattgc

SEQ ID NO: 57 - DNA, 20 bp, Artificial Sequence
  Non-complementary strand of target sequence of human DMD gene
gtagagttagaatccctgtt

SEQ ID NO: 58 - DNA, 20 bp, Artificial Sequence
  Non-complementary strand of target sequence of human DMD gene
tgactaaagtttagtcaagc

SEQ ID NO: 59 - DNA, 20 bp, Artificial Sequence
  Non-complementary strand of target sequence of human DMD gene
cattcgtattctgggtgtct

SEQ ID NO: 60 - DNA, 20 bp, Artificial Sequence
  Non-complementary strand of target sequence of human DMD gene
aagagagatacgggttaatc

SEQ ID NO: 61 - DNA, 20 bp, Artificial Sequence
  Non-complementary strand of target sequence of human DMD gene
atcaatgtcaggcatcagtc

SEQ ID NO: 62 - DNA, 20 bp, Artificial Sequence
  Non-complementary strand of target sequence of human DMD gene
ggtacaaacacagtatacga

SEQ ID NO: 63 - DNA, 20 bp, Artificial Sequence
  Non-complementary strand of target sequence of human DMD gene
tacagtctcttgcaatttac

SEQ ID NO: 64 - DNA, 20 bp, Artificial Sequence
  Non-complementary strand of target sequence of human DMD gene
attccgggtgctgcattttc

SEQ ID NO: 65 - DNA, 20 bp, Artificial Sequence
  Non-complementary strand of target sequence of human DMD gene
cagcctcagtgtaatccatt

SEQ ID NO: 66 - DNA, 20 bp, Artificial Sequence
  Non-complementary strand of target sequence of human DMD gene
atagtatatttgatctagct

SEQ ID NO: 67 - DNA, 20 bp, Artificial Sequence
  Non-complementary strand of target sequence of human DMD gene
gattgtggtcaagccatctc

SEQ ID NO: 68 - DNA, 20 bp, Artificial Sequence
  Non-complementary strand of target sequence of human DMD gene
gtatgctctgtactcagaat

SEQ ID NO: 69 - DNA, 20 bp, Artificial Sequence
  Non-complementary strand of target sequence of human DMD gene
atccttgttctgctacttac

SEQ ID NO: 70 - DNA, 20 bp, Artificial Sequence
  Non-complementary strand of target sequence of human DMD gene
ttaccaaatggattacactg

SEQ ID NO: 71 - DNA, 20 bp, Artificial Sequence
  Non-complementary strand of target sequence of human DMD gene
tttggagagcatcagattac

SEQ ID NO: 72 - DNA, 20 bp, Artificial Sequence
  Non-complementary strand of target sequence of human DMD gene
tttaaggaccacaatctcag

SEQ ID NO: 73 - DNA, 20 bp, Artificial Sequence
  Non-complementary strand of target sequence of human DMD gene
tcatcttcattggcctcatg

SEQ ID NO: 74 - DNA, 20 bp, Artificial Sequence
  Non-complementary strand of target sequence of human DMD gene
atatgatgttctaccacatg

SEQ ID NO: 75 - DNA, 20 bp, Artificial Sequence
  Non-complementary strand of target sequence of human DMD gene
gctgattttctaacgaattt

SEQ ID NO: 76 - DNA, 20 bp, Artificial Sequence
  Non-complementary strand of target sequence of human DMD gene
gctgattttctaatgaattt

SEQ ID NO: 77 - DNA, 20 bp, Artificial Sequence
  Non-complementary strand of target sequence of human DMD gene
gtttggtgattcttacggac

SEQ ID NO: 78 - DNA, 20 bp, Artificial Sequence
  Non-complementary strand of target sequence of human DMD gene
tattcattagttctggaggg

SEQ ID NO: 79 - DNA, 20 bp, Artificial Sequence
  Non-complementary strand of target sequence of human DMD gene
caactgcagcagcacgcatt

SEQ ID NO: 80 - DNA, 20 bp, Artificial Sequence
  Non-complementary strand of target sequence of human DMD gene
tgtgccttcaatacattcca

SEQ ID NO: 81 - DNA, 20 bp, Artificial Sequence
  Non-complementary strand of target sequence of human DMD gene
taatgtgtcttgtttcgtaa

SEQ ID NO: 82 - DNA, 20 bp, Artificial Sequence
  Non-complementary strand of target sequence of human DMD gene
tcatattctgtagtacaagg

SEQ ID NO: 83 - DNA, 20 bp, Artificial Sequence
  Non-complementary strand of target sequence of human DMD gene
acaaaactccataatagttc

SEQ ID NO: 84 - DNA, 20 bp, Artificial Sequence
  Non-complementary strand of target sequence of human DMD gene
ttgcatagagtacgtaacag

SEQ ID NO: 85 - DNA, 20 bp, Artificial Sequence
  Non-complementary strand of target sequence of human DMD gene
gttgaaattaaactacacac

SEQ ID NO: 86 - DNA, 20 bp, Artificial Sequence
  Non-complementary strand of target sequence of human DMD gene
tgtatgatgctataatacca

SEQ ID NO: 87 - RNA, 20 nt, Artificial Sequence
  Guide sequence based on human DMD gene
cgccugcagguaaaagcaua

SEQ ID NO: 88 - RNA, 20 nt, Artificial Sequence
  Guide sequence based on human DMD gene
aggguauuuagcaguauccc

SEQ ID NO: 89 - RNA, 20 nt, Artificial Sequence
  Guide sequence based on human DMD gene
cagcuauccucugcauugua

SEQ ID NO: 90 - RNA, 20 nt, Artificial Sequence
  Guide sequence based on human DMD gene
gccuguaacccuagcgcuua

SEQ ID NO: 91 - RNA, 20 nt, Artificial Sequence
  Guide sequence based on human DMD gene
uguaacccuagcgcuuaggg

SEQ ID NO: 92 - RNA, 20 nt, Artificial Sequence
  Guide sequence based on human DMD gene
gcgcuuagggaggccgaugu

SEQ ID NO: 93 - RNA, 20 nt, Artificial Sequence
  Guide sequence based on human DMD gene
cgaugugggcggaucacuug

SEQ ID NO: 94 - RNA, 20 nt, Artificial Sequence
  Guide sequence based on human DMD gene
caucggccucccuaagcgcu

SEQ ID NO: 95 - RNA, 20 nt, Artificial Sequence
  Guide sequence based on human DMD gene
gagaaucacuugaacgugga

SEQ ID NO: 96 - RNA, 20 nt, Artificial Sequence
  Guide sequence based on human DMD gene
gauuuuaugauuccacaauc

SEQ ID NO: 97 - RNA, 20 nt, Artificial Sequence
  Guide sequence based on human DMD gene
aaaucuccagguagguaacc

SEQ ID NO: 98 - RNA, 20 nt, Artificial Sequence
  Guide sequence based on human DMD gene
ccacuucagccucuacuuaa

SEQ ID NO: 99 - RNA, 20 nt, Artificial Sequence
  Guide sequence based on human DMD gene
caggcuucugaaccuucgcc

SEQ ID NO: 100 - RNA, 20 nt, Artificial Sequence
  Guide sequence based on human DMD gene
uugauguggccagacuauac

SEQ ID NO: 101 - RNA, 20 nt, Artificial Sequence
  Guide sequence based on human DMD gene
guagaguuagaaucccuguu

SEQ ID NO: 102 - RNA, 20 nt, Artificial Sequence
  Guide sequence based on human DMD gene
ugcuugacuaaacuuuaguc

SEQ ID NO: 103 - RNA, 20 nt, Artificial Sequence
  Guide sequence based on human DMD gene
ucuggccacaucaaauuacc

SEQ ID NO: 104 - RNA, 20 nt, Artificial Sequence
  Guide sequence based on human DMD gene
cucucuggggaagauugccc

SEQ ID NO: 105 - RNA, 20 nt, Artificial Sequence
  Guide sequence based on human DMD gene
uaacuaaccucauguguuaa

SEQ ID NO: 106 - RNA, 20 nt, Artificial Sequence
  Guide sequence based on human DMD gene
gcaaguaauaacacaagcuu

SEQ ID NO: 107 - RNA, 20 nt, Artificial Sequence
  Guide sequence based on human DMD gene
gcuuaggaaucaaauggacu

SEQ ID NO: 108 - RNA, 20 nt, Artificial Sequence
  Guide sequence based on human DMD gene
aaauucguuagaaaaucagc

SEQ ID NO: 109 - RNA, 20 nt, Artificial Sequence
  Guide sequence based on human DMD gene
aguaggaaccauauaguagc

SEQ ID NO: 110 - RNA, 20 nt, Artificial Sequence
  Guide sequence based on human DMD gene
uucuuacggacaggcagcuu

SEQ ID NO: 111 - RNA, 20 nt, Artificial Sequence
  Guide sequence based on human DMD gene
gugccuucaauacauuccaa

SEQ ID NO: 112 - RNA, 20 nt, Artificial Sequence
  Guide sequence based on human DMD gene
gacacauuaacaucaacaac

SEQ ID NO: 113 - RNA, 19 nt, Artificial Sequence
  Guide sequence based on human DMD gene
ccaccuauuauguggauga

SEQ ID NO: 114 - RNA, 20 nt, Artificial Sequence
  Guide sequence based on human DMD gene
cccaucauccacauaauagg

SEQ ID NO: 115 - RNA, 20 nt, Artificial Sequence
  Guide sequence based on human DMD gene
ccacccaucauccacauaau

SEQ ID NO: 116 - RNA, 20 nt, Artificial Sequence
  Guide sequence based on human DMD gene
uacguaacaguggcaaugua

SEQ ID NO: 117 - RNA, 20 nt, Artificial Sequence
  Guide sequence based on human DMD gene
gaaucauauucuguaguaca

SEQ ID NO: 118 - RNA, 20 nt, Artificial Sequence
  Guide sequence based on human DMD gene
aaacagugaaaacagaacgu

SEQ ID NO: 119 - DNA, 20 bp, Artificial Sequence
  Non-complementary strand of target sequence of human DMD gene
cgcctgcaggtaaaagcata

SEQ ID NO: 120 - DNA, 20 bp, Artificial Sequence
  Non-complementary strand of target sequence of human DMD gene
agggtatttagcagtatccc

SEQ ID NO: 121 - DNA, 20 bp, Artificial Sequence
  Non-complementary strand of target sequence of human DMD gene
cagctatcctctgcattgta

SEQ ID NO: 122 - DNA, 20 bp, Artificial Sequence
  Non-complementary strand of target sequence of human DMD gene
gcctgtaaccctagcgctta

SEQ ID NO: 123 - DNA, 20 bp, Artificial Sequence
  Non-complementary strand of target sequence of human DMD gene
tgtaaccctagcgcttaggg

SEQ ID NO: 124 - DNA, 20 bp, Artificial Sequence
  Non-complementary strand of target sequence of human DMD gene
gcgcttagggaggccgatgt

SEQ ID NO: 125 - DNA, 20 bp, Artificial Sequence
  Non-complementary strand of target sequence of human DMD gene
cgatgtgggcggatcacttg

SEQ ID NO: 126 - DNA, 20 bp, Artificial Sequence
  Non-complementary strand of target sequence of human DMD gene
catcggcctccctaagcgct

SEQ ID NO: 127 - DNA, 20 bp, Artificial Sequence
  Non-complementary strand of target sequence of human DMD gene
gagaatcacttgaacgtgga

SEQ ID NO: 128 - DNA, 20 bp, Artificial Sequence
  Non-complementary strand of target sequence of human DMD gene
gattttatgattccacaatc

SEQ ID NO: 129 - DNA, 20 bp, Artificial Sequence
  Non-complementary strand of target sequence of human DMD gene
aaatctccaggtaggtaacc

SEQ ID NO: 130 - DNA, 20 bp, Artificial Sequence
  Non-complementary strand of target sequence of human DMD gene
ccacttcagcctctacttaa

SEQ ID NO: 131 - DNA, 20 bp, Artificial Sequence
  Non-complementary strand of target sequence of human DMD gene
caggcttctgaaccttcgcc

SEQ ID NO: 132 - DNA, 20 bp, Artificial Sequence
  Non-complementary strand of target sequence of human DMD gene
ttgatgtggccagactatac

SEQ ID NO: 133 - DNA, 20 bp, Artificial Sequence
  Non-complementary strand of target sequence of human DMD gene
gtagagttagaatccctgtt

SEQ ID NO: 134 - DNA, 20 bp, Artificial Sequence
  Non-complementary strand of target sequence of human DMD gene
tgcttgactaaactttagtc

SEQ ID NO: 135 - DNA, 20 bp, Artificial Sequence
  Non-complementary strand of target sequence of human DMD gene
tctggccacatcaaattacc

SEQ ID NO: 136 - DNA, 20 bp, Artificial Sequence
  Non-complementary strand of target sequence of human DMD gene
ctctctggggaagattgccc

SEQ ID NO: 137 - DNA, 20 bp, Artificial Sequence
  Non-complementary strand of target sequence of human DMD gene
taactaacctcatgtgttaa

SEQ ID NO: 138 - DNA, 20 bp, Artificial Sequence
  Non-complementary strand of target sequence of human DMD gene
gcaagtaataacacaagctt

SEQ ID NO: 139 - DNA, 20 bp, Artificial Sequence
  Non-complementary strand of target sequence of human DMD gene
gcttaggaatcaaatggact

SEQ ID NO: 140 - DNA, 20 bp, Artificial Sequence
  Non-complementary strand of target sequence of human DMD gene
aaattcgttagaaaatcagc

SEQ ID NO: 141 - DNA, 20 bp, Artificial Sequence
  Non-complementary strand of target sequence of human DMD gene
agtaggaaccatatagtagc

SEQ ID NO: 142 - DNA, 20 bp, Artificial Sequence
  Non-complementary strand of target sequence of human DMD gene
ttcttacggacaggcagctt

SEQ ID NO: 143 - DNA, 20 bp, Artificial Sequence
  Non-complementary strand of target sequence of human DMD gene
gtgccttcaatacattccaa

SEQ ID NO: 144 - DNA, 20 bp, Artificial Sequence
  Non-complementary strand of target sequence of human DMD gene
gacacattaacatcaacaac

SEQ ID NO: 145 - DNA, 19 bp, Artificial Sequence
  Non-complementary strand of target sequence of human DMD gene
ccacctattatgtggatga

SEQ ID NO: 146 - DNA, 20 bp, Artificial Sequence
  Non-complementary strand of target sequence of human DMD gene
cccatcatccacataatagg

SEQ ID NO: 147 - DNA, 20 bp, Artificial Sequence
  Non-complementary strand of target sequence of human DMD gene
ccacccatcatccacataat

SEQ ID NO: 148 - DNA, 20 bp, Artificial Sequence
  Non-complementary strand of target sequence of human DMD gene
tacgtaacagtggcaatgta

SEQ ID NO: 149 - DNA, 20 bp, Artificial Sequence
  Non-complementary strand of target sequence of human DMD gene
gaatcatattctgtagtaca

SEQ ID NO: 150 - DNA, 20 bp, Artificial Sequence
  Non-complementary strand of target sequence of human DMD gene
aaacagtgaaaacagaacgt

SEQ ID NO: 151 - RNA, 64 nt, Artificial Sequence
  tracrRNA sequence
uagcaaguuaaaauaaggcuaguccguuaucaacuugaaaaaguggcaccgagucggugcuuuu

SEQ ID NO: 152 - RNA, 12 nt, Artificial Sequence
  crRNA repeat sequence
guuuuagagcua

SEQ ID NO: 153 - RNA, 80 nt, Artificial Sequence
  crRNA repeat-tracrRNA fusion sequence
guuuuagagcuagaaauagcaaguuaaaauaaggcuaguccguuaucaacuugaaaaaguggcaccgagucggugcuuuu

(19) United States
(12) Patent Application Publication
(10) Pub. No.: US 2022/0177876 A1
(43) Pub. Date: Jun. 9, 2022

(54) Compositions and Methods for Modifying Dystrophin Genes

(71) Applicant: The Regents of the University of California, Oakland, CA (US)

(72) Inventors: Melissa J. Spencer, Los Angeles, CA (US); Courtney S. Young, Los Angeles, CA (US); April D. Pyle, Los Angeles, CA (US); Michael Reza Emami, Diamond Bar, CA (US)

(21) Appl. No.: 17/441,888

(22) PCT Filed: Apr. 7, 2020
(86) PCT No.: PCT/US2020/027040

§ 371 Date: Sep. 22, 2021

Related U.S. Application Data
(60) Provisional application No. 62/833,143, filed on Apr. 12, 2019.

(51) Int. Cl. C12N 15/11; A61K 38/46

(52) U.S. Cl. CPC C12N 15/11; C12N 2310/20; A61K 38/465

(57) ABSTRACT

Disclosed herein are guide sequences for modifying a dystrophin gene using CRISPR technology. Specifically, the disclosure provides a method of modifying a dystrophin gene in a cell or a subject, which comprises introducing into the cell or subject (a) a Cas protein or a nucleotide sequence encoding the Cas protein; and a single guide RNA (gRNA), or a first gRNA and a second gRNA, wherein the Cas protein is a type II CRISPR/Cas endonuclease. Further disclosed are gRNA nucleic acid sequences.

### CROSS-REFERENCE TO RELATED APPLICATIONS

This application claims the benefit of U.S. Patent Application No. 62/833,143, filed Apr. 12, 2019, which is herein incorporated by reference in its entirety.

### REFERENCE TO A SEQUENCE LISTING SUBMITTED VIA EFS-WEB

The content of the ASCII text file of the sequence listing named “20200407_034044_201WO1_ST25” which is 33.6 kb in size was created on Apr. 6, 2020 and electronically submitted via EFS-Web herewith the application is incorporated herein by reference in its entirety.

### BACKGROUND OF THE INVENTION

### 1. Field of the Invention

The field generally relates to compositions and methods for modifying dystrophin genes.

### 2. Description of the Related Art

Dystrophin plays an important structural role in the muscle fiber, connecting the extracellular matrix and the cytoskeleton. The N-terminal region binds actin, whereas the C-terminal end maintains the dystrophin glycoprotein complex (DGC) at the sarcolemma membrane. In the absence of functional dystrophin, mechanical stress leads to sarcolemmal ruptures, causing an uncontrolled influx of calcium into the muscle fiber interior, thereby triggering calcium-activated proteases and fiber necrosis.

Duchenne muscular dystrophy (DMD) is caused by a defect in the expression of dystrophin. The gene encoding dystrophin contains 79 exons spread out over more than 2 million nucleotides of DNA. Any exonic mutation that changes the reading frame of the exon, or introduces a stop codon, or is characterized by removal of an entire out of frame exon or exons or duplications of one or more exons has the potential to disrupt production of functional dystrophin, resulting in DMD. A frameshift in the DMD gene can result in the production of a truncated non-functional dystrophin protein, resulting in progressive muscle wasting and weakness.

### SUMMARY OF THE INVENTION

In some embodiments, the present invention provides nucleic acid molecules, which comprise or consist of a sequence having 100% sequence identity to at least 17, at least 18, or at least 19 nucleotides of a sequence selected from the group consisting of SEQ ID NOs: 1-43 and 87-118 (preferably selected from the group consisting of SEQ ID NOs: 1-41 and 87-118), and compositions thereof. In some embodiments, the present invention provides guide RNAs, which comprise a nucleic acid molecule comprising or consisting of a nucleic acid molecule having 100% sequence identity to at least 17, at least 18, or at least 19 nucleotides of a sequence selected from the group consisting of SEQ ID NOs: 1-43 and 87-118 (preferably selected from the group consisting of SEQ ID NOs: 1-41 and 87-118), and compositions thereof. In some embodiments, the present invention provides nucleic acid molecules, which encode a guide RNA that comprises a nucleic acid molecule which comprises or consists of a nucleic acid molecule having 100% sequence identity to at least 17, at least 18, or at least 19 nucleotides of a sequence selected from the group consisting of SEQ ID NOs: 1-43 and 87-118 (preferably selected from the group consisting of SEQ ID NOs: 1-41 and 87-118), and compositions thereof.

In some embodiments, the present invention provides kits comprising one or more nucleic acid molecules, which comprise or consist of a sequence having 100% sequence identity to at least 17, at least 18, or at least 19 nucleotides of a sequence selected from the group consisting of SEQ ID NOs: 1-43 and 87-118 (preferably selected from the group consisting of SEQ ID NOs: 1-41 and 87-118), and/or one or more compositions thereof. In some embodiments, the present invention provides kits comprising one or more guide RNAs, which comprise a nucleic acid molecule comprising or consisting of a nucleic acid molecule having 100% sequence identity to at least 17, at least 18, or at least 19 nucleotides of a sequence selected from the group consisting of SEQ ID NOs: 1-43 and 87-118 (preferably selected from the group consisting of SEQ ID NOs: 1-41 and 87-118), and/or one or more compositions thereof. In some embodiments, the present invention provides one or more nucleic acid molecules, which encode a guide RNA that comprises a nucleic acid molecule which comprises or consists of a nucleic acid molecule having 100% sequence identity to at least 17, at least 18, or at least 19 nucleotides of a sequence selected from the group consisting of SEQ ID NOs: 1-43 and 87-118 (preferably selected from the group consisting of SEQ ID NOs: 1-41 and 87-118), and/or one or more compositions thereof.

In some embodiments, the present invention provides methods for modifying a dystrophin gene in a cell or a subject, which comprise introducing into the cell or subject (a) a Cas protein or a nucleotide sequence encoding the Cas protein; and (b) at least one guide RNA, which comprises a nucleic acid molecule comprising or consisting of a nucleic acid molecule having 100% sequence identity to at least 17, at least 18, or at least 19 nucleotides of a sequence selected from the group consisting of SEQ ID NOs: 1-43 and 87-118 (preferably selected from the group consisting of SEQ ID NOs: 1-41 and 87-118), or at least one nucleic acid molecule that encodes the at least one guide RNA. In some embodiments, the Cas protein is a class 2 CRISPR/Cas endonuclease. In some embodiments, the Cas protein is a type II CRISPR/Cas endonuclease. In some embodiments, the Cas protein is a Cas9 endonuclease. In some embodiments, the cell is a muscle cell, a pericyte, an induced pluripotent stem (iPS) cell, or a stem cell. In some embodiments, the cell is a human cell and/or the subject is human. In some embodiments, the cell is genetically modified to have the dystrophin gene and/or the subject is an animal genetically modified to have the dystrophin gene.

In some embodiments, the present invention provides a method of treating Duchenne muscular dystrophy or Becker muscular dystrophy in a subject, which comprises modifying a dystrophin gene in a cell or a subject, which comprise introducing into the cell or subject (a) a Cas protein or a nucleotide sequence encoding the Cas protein; and (b) at least one guide RNA, which comprises a nucleic acid molecule comprising or consisting of a nucleic acid molecule having 100% sequence identity to at least 17, at least 18, or at least 19 nucleotides of a sequence selected from the group consisting of SEQ ID NOs: 1-43 and 87-118 (preferably selected from the group consisting of SEQ ID NOs: 1-41 and 87-118), or at least one nucleic acid molecule that encodes the at least one guide RNA. In some embodiments, the Cas protein is a class 2 CRISPR/Cas endonuclease. In some embodiments, the Cas protein is a type II CRISPR/Cas endonuclease. In some embodiments, the Cas protein is a Cas9 endonuclease. In some embodiments, the cell is a muscle cell, a pericyte, an induced pluripotent stem (iPS) cell, or a stem cell. In some embodiments, the cell is a human cell and/or the subject is human. In some embodiments, the cell is genetically modified to have the dystrophin gene and/or the subject is an animal genetically modified to have the dystrophin gene. In some embodiments, the dystrophin gene in the cell is modified ex vivo and then the cell is administered to the subject. In some embodiments, the dystrophin gene in the cell is modified in vivo in the subject.

Both the foregoing general description and the following detailed description are exemplary and explanatory only and are intended to provide further explanation of the invention as claimed. The accompanying drawings are included to provide a further understanding of the invention and are incorporated in and constitute part of this specification, illustrate several embodiments of the invention, and together with the description explain the principles of the invention.

### DESCRIPTION OF THE DRAWINGS

This invention is further understood by reference to the drawings wherein:

FIG. 1 schematically shows different strategies for reframing the DMD gene. Shown is a cartoon of the DMD gene (not to scale) with an example DMD out-of-frame mutation. The different reframing strategies and distance to be covered in a wildtype gene is listed. Distance G is a reframing strategy disclosed in PCT/US2017/017255.

FIG. 2 schematically shows additional strategies for reframing the DMD gene. Shown is a cartoon of the DMD gene (not to scale) with an example DMD out-of-frame mutation. The different reframing strategies and distance to be covered in a wildtype gene is listed. Again, distance G is a reframing strategy disclosed in PCT/US2017/017255.

FIG. 3 shows the qPCR efficiency of an exon 46 deletion across different strategies. The efficiency of deletion, measured by 1 minus the fold change compared to GFP control undeleted 293FT cells, was assessed by qPCR across different strategies and their corresponding deletion size. Average±standard error of the mean is shown.

FIG. 4 shows the restoration of dystrophin in vitro after transfection of different strategies. 1003 myoblasts were transfected with the different CRISPR strategies then differentiated to myotubes for 5 days. They were stained for myosin heavy chain (MyHC, middle panels) to mark muscle cells and dystrophin (right panels). Color was inverted and then converted to grayscale.

FIG. 5 shows the restoration of dystrophin in vivo after local adeno-associated virus (AAV) 9 delivery of some of the different CRISPR strategies to the tibialis anterior (TA) muscle i.m. in hDMD del45 mice. Panel a) Staining with laminin to mark muscle fibers (first column), dystrophin to mark reframed cells (second column) and dystrophin 48/50B to mark revertant fibers since this epitope is missing in some of the strategies (third column) is shown. Color was inverted and then converted to grayscale. Panel b) The number of dystrophin-positive fibers was quantified.

FIG. 6 shows the restoration of dystrophin in vivo in the heart after systemic AAV9 delivery of some of the different CRISPR strategies in hDMD del45 mice. Dystrophin is assessed through Western blotting for human dystrophin protein (Panel A) with the percent wild type dystrophin restored (Panel B) and immunostaining with dystrophin (Panel C, color was inverted and then converted to grayscale).

### DETAILED DESCRIPTION

Disclosed herein are guide sequences for guide RNAs (gRNAs) for reframing a DMD gene using CRISPR technology to treat Duchenne muscular dystrophy. The guide sequences of the gRNAs and the sequences of the non-complementary strand of their target sequences are set forth in Table 1 and additional ones are set forth in Table 2:

TABLE 1
gRNA Guide Sequences and Efficiencies

| | | | | |
|---|---|---|---|---|
| | | | Average | Average |
| | | | Efficiency ± | Efficiency ± |
| | | Non-Complementary Strand | SD by TIDE in | SD by TIDE in |
| Name | Guide Sequence | of Target Sequence | 293FT (%) | 293AAV (%) |

gRNA target position 1

| | | | | |
|---|---|---|---|---|
| 44FS1 | AGAUCUGUCAAAUCGCCUGC | AGATCTGTCAAATCGCCTGC | 6.75 ± 0.49 | nd |
| | (SEQ ID NO: 1) | (SEQ ID NO: 44) | | |
| 44FS2 | AUCCAUAUGCUUUUACCUGC | ATCCATATGCTTTTACCTGC | 1.5 ± 0.57 | nd |
| | (SEQ ID NO: 2) | (SEQ ID NO: 45) | | |

gRNA target position 5

| | | | | |
|---|---|---|---|---|
| 46FS1 | AUUCUUUUGUUCUUCUAGCC | ATTCTTTTGTTCTTCTAGCC | 0.6 ± 0.28 | nd |
| | (SEQ ID NO: 3) | (SEQ ID NO: 46) | | |
| 46FS2 | AAUUUUAUUCUUCUUUCUCC | AATTTTATTCTTCTTTCTCC | 3 ± 0 | nd |
| | (SEQ ID NO: 4) | (SEQ ID NO: 47) | | |

gRNA target position 4

| | | | | |
|---|---|---|---|---|
| 45int1 | AUCGGCCUCCCUAAGCGCUA | ATCGGCCTCCCTAAGCGCTA | 10.8 ± 2.55 | nd |
| | (SEQ ID NO: 5) | (SEQ ID NO: 48) | | |
| 45int2 | CUCAAGUGAUCCGCCCACAU | CTCAAGTGATCCGCCCACAT | 2.8 ± 1.41 | nd |
| | (SEQ ID NO: 6) | (SEQ ID NO: 49) | | |

gRNA target position 6

| | | | | |
|---|---|---|---|---|
| 46int1 | AUCUGUCCUGGUUACCUACC | ATCTGTCCTGGTTACCTACC | 0.4 ± 0.42 | nd |
| | (SEQ ID NO: 7) | (SEQ ID NO: 50) | | |
| 46int2 | CUGAAGUGGUGAGACUGAUC | CTGAAGTGGTGAGACTGATC | 5.9 ± 1.41 | nd |
| | (SEQ ID NO: 8) | (SEQ ID NO: 51) | | |
| 46int3 | GUGCACUGGUUCAGAACUGC | GTGCACTGGTTCAGAACTGC | 3.15 ± 0.07 | nd |
| | (SEQ ID NO: 9) | (SEQ ID NO: 52) | | |

gRNA target position 7

| | | | | |
|---|---|---|---|---|
| 48SC16 | AGGCUUCUGAACCUUCGCCU | AGGCTTCTGAACCTTCGCCT | 0.45 ± .07 | 0.85 ± 0.07 |
| | (SEQ ID NO: 10) | (SEQ ID NO: 53) | | |
| 48SC2 | AGCAAUCUCCCCGUAUAGUC | AGCAATCTCCCCGTATAGTC | nd | 0.3 ± 0.07 |
| | (SEQ ID NO: 11) | (SEQ ID NO: 54) | | |
| 48SC3 | UGAUGUGGCCAGACUAUACG | TGATGTGGCCAGACTATACG | 0.35 ± 0.21 | 0.75 ± .78 |
| | (SEQ ID NO: 12) | (SEQ ID NO: 55) | | |
| 48SC4 | AGACUAUACGGGGAGAUUGC | AGACTATACGGGGAGATTGC | 1.15 ± .64 | 0.55 ± .35 |
| | (SEQ ID NO: 13) | (SEQ ID NO: 56) | | |
| 48SC5 | GUAGAGUUAGAAUCCCUGUU | GTAGAGTTAGAATCCCTGTT | nd | 0.35 ± .07 |
| | (SEQ ID NO: 14) | (SEQ ID NO: 57) | | |
| 48SC11 | UGACUAAAGUUUAGUCAAGC | TGACTAAAGTTTAGTCAAGC | 0.85 ± 0.5 | 0.55 ± .49 |
| | (SEQ ID NO: 15) | (SEQ ID NO: 58) | | |
| 48SC12 | CAUUCGUAUUCUGGGUGUCU | CATTCGTATTCTGGGTGTCT | 7.15 ± 3.04 | 21.4 ± 21.8 |
| | (SEQ ID NO: 16) | (SEQ ID NO: 59) | | |

gRNA target position 8

| | | | | |
|---|---|---|---|---|
| 48FC6 | AAGAGAGAUACGGGUUAAUC | AAGAGAGATACGGGTTAATC | 1.5 ± 1.56 | 0.65 ± .35 |
| | (SEQ ID NO: 17) | (SEQ ID NO: 60) | | |
| 48FC7 | AUCAAUGUCAGGCAUCAGUC | ATCAATGTCAGGCATCAGTC | 0.4 ± 0.42 | 1.5 ± .71 |
| | (SEQ ID NO: 18) | (SEQ ID NO: 61) | | |
| 48FC8 | GGUACAAACACAGUAUACGA | GGTACAAACACAGTATACGA | 11.95 ± 1.2 | 7.6 ± 2.69 |
| | (SEQ ID NO: 19) | (SEQ ID NO: 62) | | |
| 48FC9 | UACAGUCUCUUGCAAUUUAC | TACAGTCTCTTGCAATTTAC | nd | 0.25 ± .07 |
| | (SEQ ID NO: 20) | (SEQ ID NO: 63) | | |
| 48FC10 | AUUCCGGGUGCUGCAUUUUC | ATTCCGGGTGCTGCATTTTC | 1.45 ± 0.49 | 0.1 ± 0 |
| | (SEQ ID NO: 21) | (SEQ ID NO: 64) | | |

gRNA target position 9

| | | | | |
|---|---|---|---|---|
| 51SC1 | CAGCCUCAGUGUAAUCCAUU | CAGCCTCAGTGTAATCCATT | 5.8 ± 1.84 | 4.2 ± 1.41 |
| | (SEQ ID NO: 22) | (SEQ ID NO: 65) | | |
| 51SC2 | AUAGUAUAUUUGAUCUAGCU | ATAGTATATTTGATCTAGCT | 1.9 ± 0.57 | 2.3 ± 0 |
| | (SEQ ID NO: 23) | (SEQ ID NO: 66) | | |
| 51SC3 | GAUUGUGGUCAAGCCAUCUC | GATTGTGGTCAAGCCATCTC | nd | 1.9 ± .42 |
| | (SEQ ID NO: 24) | (SEQ ID NO: 67) | | |
| 51SC4 | GUAUGCUCUGUACUCAGAAU | GTATGCTCTGTACTCAGAAT | 8.9 ± 0 | 2.45 ± 1.77 |
| | (SEQ ID NO: 25) | (SEQ ID NO: 68) | | |
| 51SC5 | AUCCUUGUUCUGCUACUUAC | ATCCTTGTTCTGCTACTTAC | nd | 1.5 ± .28 |
| | (SEQ ID NO: 26) | (SEQ ID NO: 69) | | |
| 51SC13 | UUACCAAAUGGAUUACACUG | TTACCAAATGGATTACACTG | 11.1 ± 2.55 | 4.9 ± 0 |
| | (SEQ ID NO: 27) | (SEQ ID NO: 70) | | |

gRNA target position 10

| | | | | |
|---|---|---|---|---|
| 51FC6 | UUUGGAGAGCAUCAGAUUAC | TTTGGAGAGCATCAGATTAC | 0.35 ± 0.07 | 1.85 ± .49 |
| | (SEQ ID NO: 28) | (SEQ ID NO: 71) | | |
| 51FC7 | UUUAAGGACCACAAUCUCAG | TTTAAGGACCACAATCTCAG | n/a | n/a |
| | (SEQ ID NO: 29) | (SEQ ID NO: 72) | | |
| 51FC8 | UCAUCUUCAUUGGCCUCAUG | TCATCTTCATTGGCCTCATG | nd | 1.65 ± .21 |
| | (SEQ ID NO: 30) | (SEQ ID NO: 73) | | |
| 51FC9 | AUAUGAUGUUCUACCACAUG | ATATGATGTTCTACCACATG | 0.05 ± 0.07 | 0.9 ± .85 |
| | (SEQ ID NO: 31) | (SEQ ID NO: 74) | | |
| 51FC10 | GCUGAUUUUCUAACGAAUUU | GCTGATTTTCTAACGAATTT | n/a | n/a |
| | (SEQ ID NO: 32) | (SEQ ID NO: 75) | | |
| 51FC10SNP | GCUGAUUUUCUAAUGAAUUU | GCTGATTTTCTAATGAATTT | 0.1 ± 0 | 0.35 ± .21 |
| | (SEQ ID NO: 33) | (SEQ ID NO: 76) | | |
| 51FC11 | GUUUGGUGAUUCUUACGGAC | GTTTGGTGATTCTTACGGAC | 3.3 ± 1.98 | 2.5 ± .71 |
| | (SEQ ID NO: 34) | (SEQ ID NO: 77) | | |
| 51FC12 | UAUUCAUUAGUUCUGGAGGG | TATTCATTAGTTCTGGAGGG | 11.25 ± 3.32 | 4.3 ± .28 |
| | (SEQ ID NO: 35) | (SEQ ID NO: 78) | | |

gRNA target position 3

| | | | | |
|---|---|---|---|---|
| 44int1 | CAACUGCAGCAGCACGCAUU | CAACTGCAGCAGCACGCATT | 5.9 ± 0.42 | nd |
| | (SEQ ID NO: 36) | (SEQ ID NO: 79) | | |
| 44int2 | UGUGCCUUCAAUACAUUCCA | TGTGCCTTCAATACATTCCA | 8.65 ± 02.76 | nd |
| | (SEQ ID NO: 37) | (SEQ ID NO: 80) | | |
| 44int3 | UAAUGUGUCUUGUUUCGUAA | TAATGTGTCTTGTTTCGTAA | 5.4 ± 0 | nd |
| | (SEQ ID NO: 38) | (SEQ ID NO: 81) | | |

gRNA target position 11

| | | | | |
|---|---|---|---|---|
| 55int1 | UCAUAUUCUGUAGUACAAGG | TCATATTCTGTAGTACAAGG | 18.25 ± 2.19 | nd |
| | (SEQ ID NO: 39) | (SEQ ID NO: 82) | | |
| 55int2 | ACAAAACUCCAUAAUAGUUC | ACAAAACTCCATAATAGTTC | 5.3 ± 2.4 | nd |
| | (SEQ ID NO: 40) | (SEQ ID NO: 83) | | |
| 55int3 | UUGCAUAGAGUACGUAACAG | TTGCATAGAGTACGTAACAG | 7.75 ± 0.35 | nd |
| | (SEQ ID NO: 41) | (SEQ ID NO: 84) | | |

gRNA target position 2

| | | |
|---|---|---|
| 44C4 | GUUGAAAUUAAACUACACAC | GTTGAAATTAAACTACACAC |
| | (SEQ ID NO: 42) | (SEQ ID NO: 85) |

gRNA target position 12

| | | |
|---|---|---|
| 55C3 | UGUAUGAUGCUAUAAUACCA | TGTATGATGCTATAATACCA |
| | (SEQ ID NO: 43) | (SEQ ID NO: 86) |

n/a = not applicable, nd = no data

TABLE 2
Additional gRNA Guide Sequences

| | | | | |
|---|---|---|---|---|
| | | | Average | Average |
| | | Non-Complementary | Efficiency ± | Efficiency ± |
| | | Strand of | SD by TIDE in | SD by TIDE in |
| Name | Guide Sequence | Target Sequence | 293FT (%) | 293AAV (%) |

gRNA target position 1

| | | | | |
|---|---|---|---|---|
| 44FS3 | CGCCUGCAGGUAAAAGCAUA | CGCCTGCAGGTAAAAGCATA | nd | nd |
| | (SEQ ID NO: 87) | (SEQ ID NO: 119) | | |

gRNA target position 4

| | | | | |
|---|---|---|---|---|
| 45int3 | AGGGUAUUUAGCAGUAUCCC | AGGGTATTTAGCAGTATCCC | nd | nd |
| | (SEQ ID NO: 88) | (SEQ ID NO: 120) | | |
| 45int4 | CAGCUAUCCUCUGCAUUGUA | CAGCTATCCTCTGCATTGTA | nd | nd |
| | (SEQ ID NO: 89) | (SEQ ID NO: 121) | | |
| 45int5 | GCCUGUAACCCUAGCGCUUA | GCCTGTAACCCTAGCGCTTA | nd | nd |
| | (SEQ ID NO: 90) | (SEQ ID NO: 122) | | |
| 45int6 | UGUAACCCUAGCGCUUAGGG | TGTAACCCTAGCGCTTAGGG | nd | nd |
| | (SEQ ID NO: 91) | (SEQ ID NO: 123) | | |
| 45int7 | GCGCUUAGGGAGGCCGAUGU | GCGCTTAGGGAGGCCGATGT | nd | nd |
| | (SEQ ID NO: 92) | (SEQ ID NO: 124) | | |
| 45int8 | CGAUGUGGGCGGAUCACUUG | CGATGTGGGCGGATCACTTG | nd | nd |
| | (SEQ ID NO: 93) | (SEQ ID NO: 125) | | |
| 45int9 | CAUCGGCCUCCCUAAGCGCU | CATCGGCCTCCCTAAGCGCT | nd | nd |
| | (SEQ ID NO: 94) | (SEQ ID NO: 126) | | |
| 45int10 | GAGAAUCACUUGAACGUGGA | GAGAATCACTTGAACGTGGA | nd | nd |
| | (SEQ ID NO: 95) | (SEQ ID NO: 127) | | |

gRNA target position 6

| | | | | |
|---|---|---|---|---|
| 46int4 | GAUUUUAUGAUUCCACAAUC | GATTTTATGATTCCACAATC | nd | nd |
| | (SEQ ID NO: 96) | (SEQ ID NO: 128) | | |
| 46int5 | AAAUCUCCAGGUAGGUAACC | AAATCTCCAGGTAGGTAACC | nd | nd |
| | (SEQ ID NO: 97) | (SEQ ID NO: 129) | | |
| 46int6 | CCACUUCAGCCUCUACUUAA | CCACTTCAGCCTCTACTTAA | nd | nd |
| | (SEQ ID NO: 98) | (SEQ ID NO: 130) | | |

gRNA target position 7

| | | | | |
|---|---|---|---|---|
| 48SC13 | CAGGCUUCUGAACCUUCGCC | CAGGCTTCTGAACCTTCGCC | nd | nd |
| | (SEQ ID NO: 99) | (SEQ ID NO: 131) | | |
| 48SC14 | UUGAUGUGGCCAGACUAUAC | TTGATGTGGCCAGACTATAC | nd | nd |
| | (SEQ ID NO: 100) | (SEQ ID NO: 132) | | |
| 48SC15 | GUAGAGUUAGAAUCCCUGUU | GTAGAGTTAGAATCCCTGTT | nd | nd |
| | (SEQ ID NO: 101) | (SEQ ID NO: 133) | | |
| 48SC16 | UGCUUGACUAAACUUUAGUC | TGCTTGACTAAACTTTAGTC | nd | nd |
| | (SEQ ID NO: 102) | (SEQ ID NO: 134) | | |
| 48SC17 | UCUGGCCACAUCAAAUUACC | TCTGGCCACATCAAATTACC | nd | nd |
| | (SEQ ID NO: 103) | (SEQ ID NO: 135) | | |

gRNA target position 8

| | | | | |
|---|---|---|---|---|
| 48FC18 | CUCUCUGGGGAAGAUUGCCC | CTCTCTGGGGAAGATTGCCC | nd | nd |
| | (SEQ ID NO: 104) | (SEQ ID NO: 136) | | |
| 48FC19 | UAACUAACCUCAUGUGUUAA | TAACTAACCTCATGTGTTAA | nd | nd |
| | (SEQ ID NO: 105) | (SEQ ID NO: 137) | | |

gRNA target position 9

| | | | | |
|---|---|---|---|---|
| 51SC14 | GCAAGUAAUAACACAAGCUU | GCAAGTAATAACACAAGCTT | nd | nd |
| | (SEQ ID NO: 106) | (SEQ ID NO: 138) | | |
| 51SC15 | GCUUAGGAAUCAAAUGGACU | GCTTAGGAATCAAATGGACT | nd | nd |
| | (SEQ ID NO: 107) | (SEQ ID NO: 139) | | |

gRNA target position 10

| | | | | |
|---|---|---|---|---|
| 51FC16 | AAAUUCGUUAGAAAAUCAGC | AAATTCGTTAGAAAATCAGC | nd | nd |
| | (SEQ ID NO: 108) | (SEQ ID NO: 140) | | |
| 51FC17 | AGUAGGAACCAUAUAGUAGC | AGTAGGAACCATATAGTAGC | nd | nd |
| | (SEQ ID NO: 109) | (SEQ ID NO: 141) | | |
| 51FC18 | UUCUUACGGACAGGCAGCUU | TTCTTACGGACAGGCAGCTT | nd | nd |
| | (SEQ ID NO: 110) | (SEQ ID NO: 142) | | |

gRNA target position 3

| | | | | |
|---|---|---|---|---|
| 44int4 | GUGCCUUCAAUACAUUCCAA | GTGCCTTCAATACATTCCAA | nd | nd |
| | (SEQ ID NO: 111) | (SEQ ID NO: 143) | | |
| 44int5 | GACACACUUAACAUCAACAAC | GACACATTAACATCAACC | nd | nd |
| | (SEQ ID NO: 112) | (SEQ ID NO: 144) | | |
| 44int6 | CCACCUAUUAUGUGGAUGA | CCACCTATTATGTGGATGA | nd | nd |
| | (SEQ ID NO: 113) | (SEQ ID NO: 145) | | |
| 44int7 | CCCAUCAUCCACAUAAUAGG | CCCATCATCCACATAATAGG | nd | nd |
| | (SEQ ID NO: 114) | (SEQ ID NO: 146) | | |
| 44int8 | CCACCCAUCAUCCACAUAAU | CCACCCATCATCCACATAAT | nd | nd |
| | (SEQ ID NO: 115) | (SEQ ID NO: 147) | | |

gRNA target position 11

| | | | | |
|---|---|---|---|---|
| 55int4 | UACGUAACAGUGGCAAUGUA | TACGTAACAGTGGCAATGTA | nd | nd |
| | (SEQ ID NO: 116) | (SEQ ID NO: 148) | | |
| 55int5 | GAAUCAUAUUCUGUAGUACA | GAATCATATTCTGTAGTACA | nd | nd |
| | (SEQ ID NO: 117) | (SEQ ID NO: 149) | | |
| 55int6 | AAACAGUGAAAACAGAACGU | AAACAGTGAAAACAGAACGT | nd | nd |
| | (SEQ ID NO: 118) | (SEQ ID NO: 150) | | |

n/a = not applicable, nd = no data

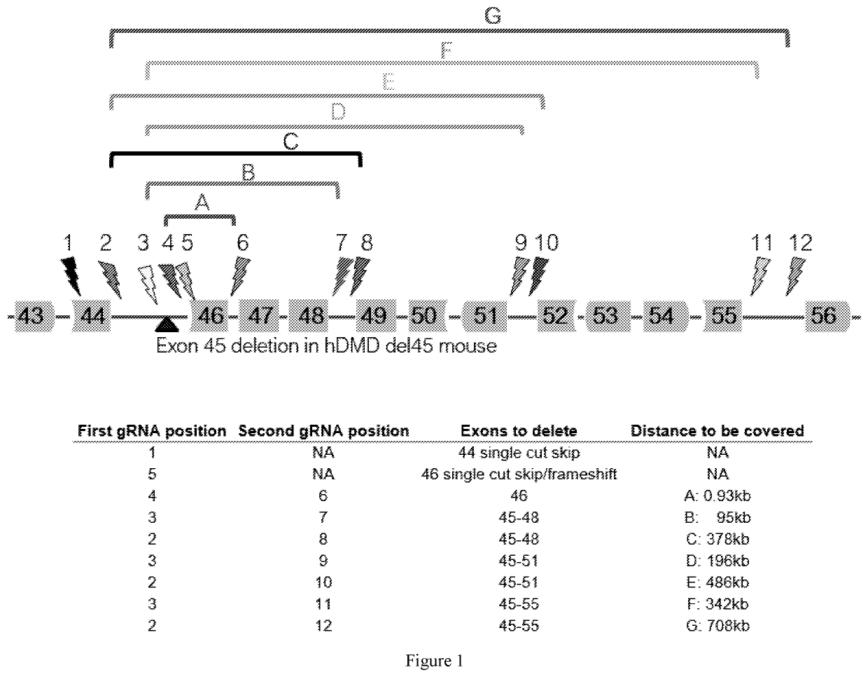

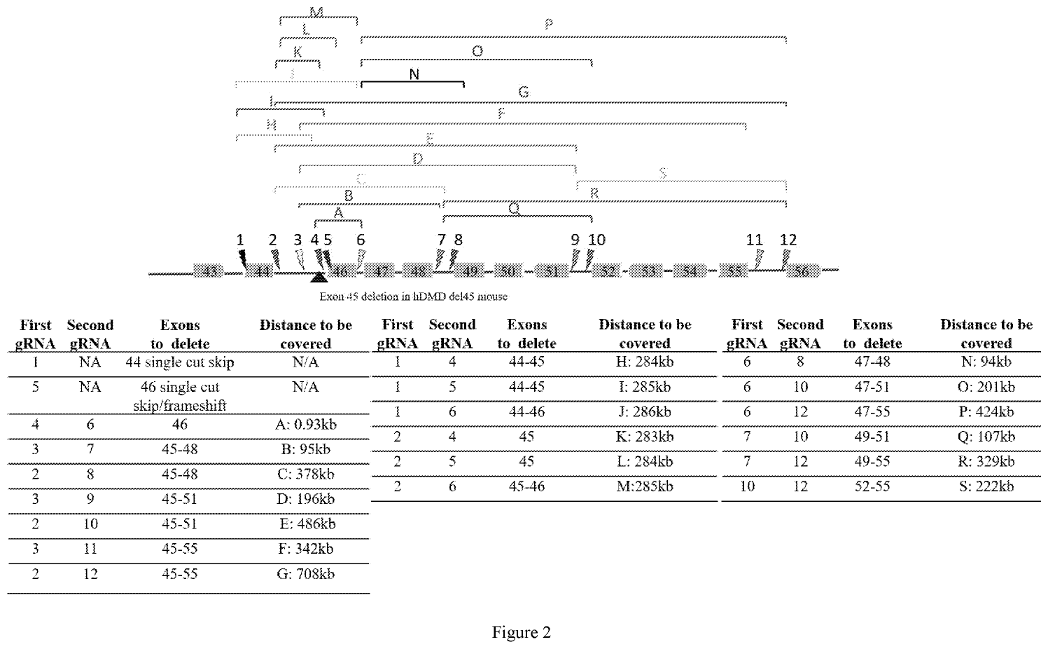

As disclosed herein, single gRNAs may be used to disrupt splice sites in a DMD gene or induce frameshifts and different combinations of two gRNAs may be used to result in a variety of deletions of different sizes in the DMD gene. Exemplary disruptions and deletions are shown in FIG. 1 and FIG. 2. While any combination of two of the indicated gRNA positions are possible to give a deletion spanning between the two selected gRNA positions, exemplary deletions A to S are shown in FIG. 1 and FIG. 2. The gRNA target position numbering, i.e., 1 to 12, in FIG. 1 and FIG. 2 corresponds to that of Tables 1 and 2. That is, if the gRNA target position is 1 the gRNA sequence is SEQ ID NO: 1, SEQ ID NO: 2, or SEQ ID NO: 87; if the gRNA target position is 2 the gRNA sequence is SEQ ID NO: 42; if the gRNA target position is 3 the gRNA sequence is SEQ ID NO: 36, SEQ ID NO: 37, SEQ ID NO: 38, SEQ ID NO: 111, SEQ ID NO: 112, SEQ ID NO: 113, SEQ ID NO: 114, or SEQ ID NO: 115; if the gRNA target position is 4 the gRNA sequence is SEQ ID NO: 5, SEQ ID NO: 6, SEQ ID NO: 88, SEQ ID NO: 89, SEQ ID NO: 90, SEQ ID NO: 91, SEQ ID NO: 92, SEQ ID NO: 93, SEQ ID NO: 94, or SEQ ID NO: 95; if the gRNA target position is 5 the gRNA sequence is SEQ ID NO: 3, or SEQ ID NO: 4; if the gRNA target position is 6 the gRNA sequence is SEQ ID NO: 7, SEQ ID NO: 8, SEQ ID NO: 9, SEQ ID NO: 96, SEQ ID NO: 97, or SEQ ID NO: 98; if the gRNA target position is 7 the gRNA sequence is SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 99, SEQ ID NO: 100, SEQ ID NO: 101, SEQ ID NO: 102, or SEQ ID NO: 103; if the gRNA target position is 8 the gRNA sequence is SEQ ID NO: 17, SEQ ID NO: 18, SEQ ID NO: 19, SEQ ID NO: 20, SEQ ID NO: 21, SEQ ID NO: 104, or SEQ ID NO: 105; if the gRNA target position is 9 the gRNA sequence is SEQ ID NO: 22, SEQ ID NO: 23, SEQ ID NO: 24, SEQ ID NO: 25, SEQ ID NO: 26, SEQ ID NO: 27, SEQ ID NO: 106, or SEQ ID NO: 107; if the gRNA target position is 10 the gRNA sequence is SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 108, SEQ ID NO: 109, or SEQ ID NO: 110; if the gRNA target position is 11 the gRNA sequence is SEQ ID NO: 39, SEQ ID NO: 40, SEQ ID NO: 41, SEQ ID NO: 116, SEQ ID NO: 117, or SEQ ID NO: 118; and if the gRNA target position is 12 the gRNA sequence is SEQ ID NO: 43.

Each guide sequence of Table 1 was cloned into px330 and screened in duplicate in HEK293FT cells by transfection with ViaFect similar to the method disclosed in Young, et al. (2016) Cell Stem Cell 18:533-40. One set was also tested in HEK293AAV cells. 3 days post administration, gDNA was isolated and PCR around the cutsite was performed. TIDE analysis to determine the efficiency of cutting was done by sequencing and analysis using methods in the art. See, e.g., Brinkman, et al. (2014) Nucleic Acids Res 42(22): e168. The percent efficiencies are shown in Table 1 and the bolded guide sequences were chosen as the most efficient ones to proceed with for each target site.

The most efficient guide sequences were cloned into px333 in pairs to create the deletions indicated in Table 3:

TABLE 3

| | First guide | Second guide |
|---|---|---|
| Deletion | sequence | sequence |
| 46delA | 45int1 | 46int2 |
| 4555S | 44int2 | 55int1 |
| 4548S | 44int2 | 48SC12 |
| 4551S | 44int2 | 51SC13 |
| 4548F | 44C4 | 48FC8 |
| 4551F | 44C4 | 51FC12 |
| 4555F | 44C4 | 55C3 |

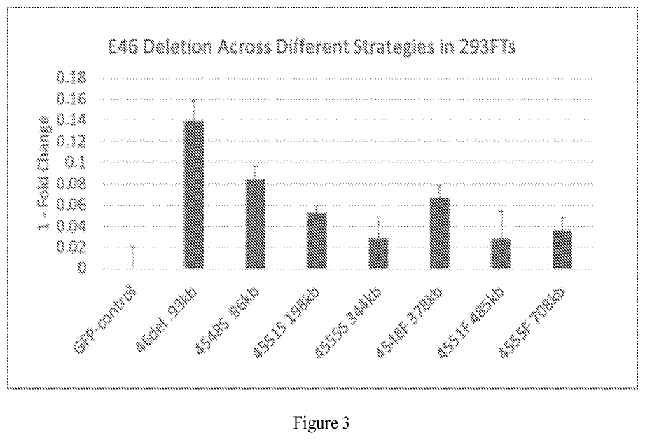

HEK293FTs were transfected with the plasmid using ViaFect in triplicate and harvested 3 days later. qPCR using TaqMan probes for DMD exon 17 (control) and DMD exon 46 (deleted with all strategies) was performed to assess the efficiency of deletion in triplicate. The percent deletion across different strategies is shown in FIG. 3. The data show the smallest size, an exon 46 deletion (0.96 kb), was the most efficient but all strategies resulted in their intended deletion.

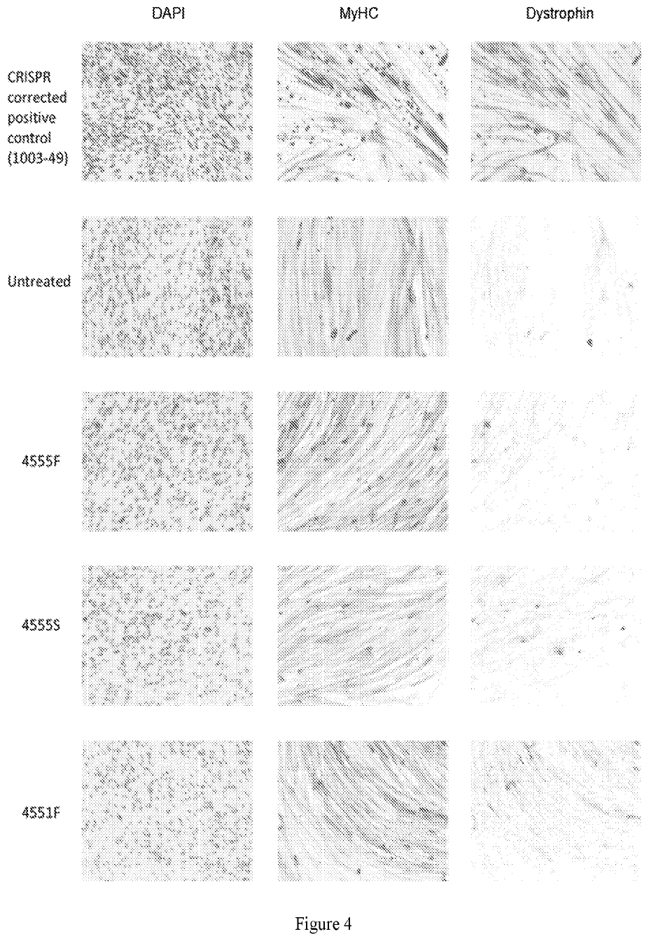

To test the ability of the different strategies to restore dystrophin, CDMD 1003 hiPSCs differentiated to myoblasts and sorted for ERBB3+ NGFR+ (Hicks, et al. (2017) Nat Cell Biol 20:46-5) were transfected with px333 containing the different strategies using Lipofectamine 2000. They were then differentiated to myotubes in low glucose DMEM with 2% horse serum and 1% ITS for 5 days. Some were harvested for protein extraction and some for immunostaining. Immunostaining for human dystrophin using MANDYS106 was performed (Young, et al., (2016) Cell Stem Cell 18:533-40). As shown in FIG. 4, there is some restored dystrophin observed in the treated samples demonstrating these different strategies can reframe the DMD gene and restore the protein. Due to the underlying mutation in this patient cell line (exon 46-51 deletion), only 3 strategies were tested.

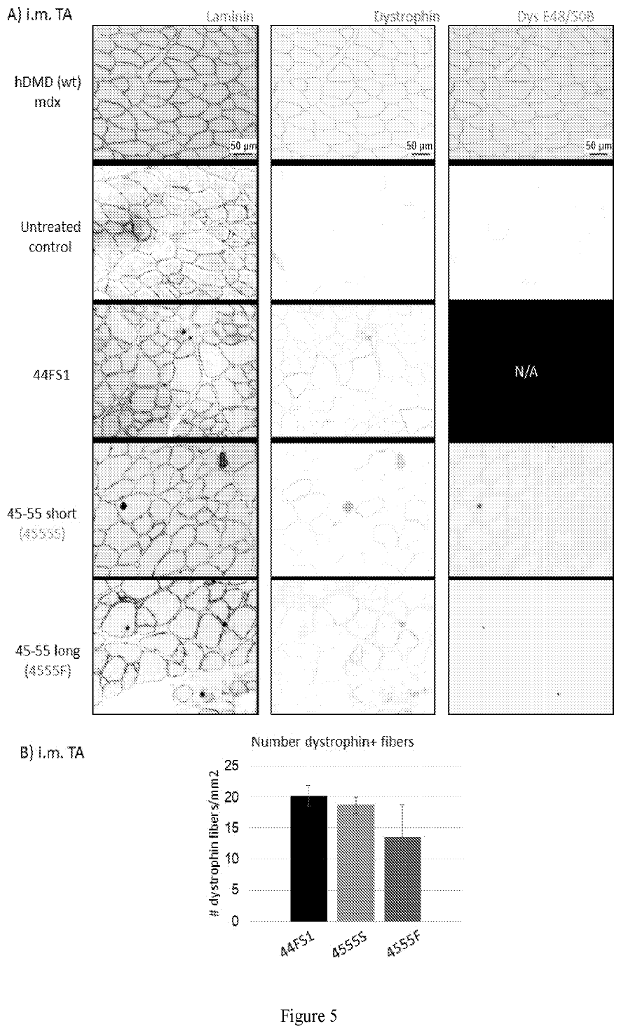

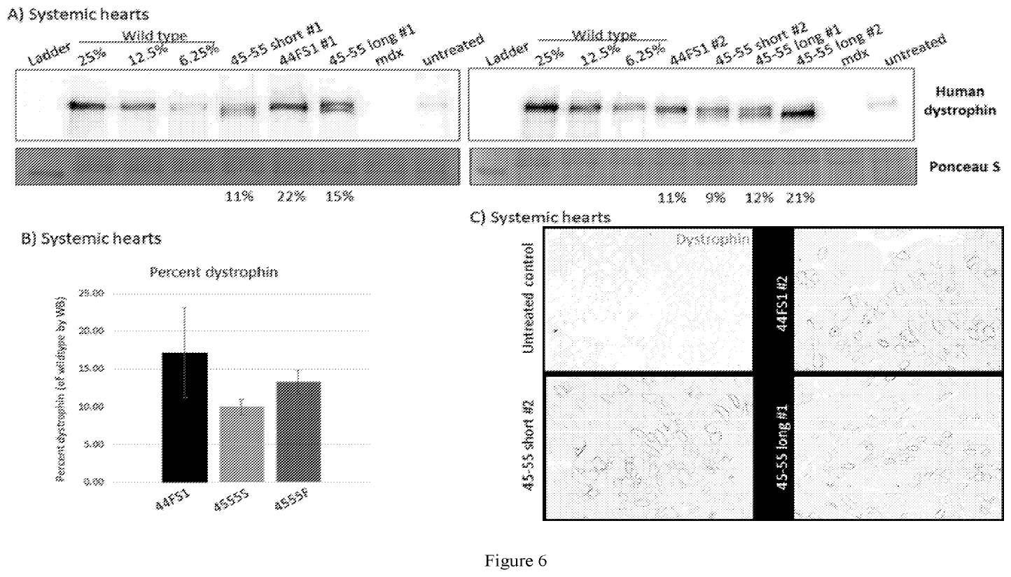

The ability of some of the strategies to restore dystrophin in vivo was tested using adeno-associated virus 9 (AAV) delivery of the therapy to hDMD del45 mice locally and systemically. As shown in FIG. 5 and FIG. 6, there is some restored dystrophin observed in the treated samples demonstrating all of the tested strategies can reframe the DMD gene and restore the protein in vivo.

Therefore, the present invention provides guide sequences, gRNAs, compositions, kits, and methods for modifying a dystrophin gene in the genome of a cell.

### Guide Sequences

In some embodiments, the present invention is directed to guide sequences for guide RNAs (gRNAs). In some embodiments, the guide sequences comprise or consist of a sequence having 100% sequence identity to at least 17 nucleotides of a sequence selected from the group consisting of SEQ ID NOs: 1-43 and 87-118 (preferably selected from the group consisting of SEQ ID NOs: 1-41 and 87-118). In some embodiments, the guide sequences comprise or consist of a sequence having 100% sequence identity to at least 17 contiguous nucleotides of a sequence selected from the group consisting of SEQ ID NOs: 1-43 and 87-118 (preferably selected from the group consisting of SEQ ID NOs: 1-41 and 87-118). In some embodiments, the guide sequences comprise or consist of a sequence having 100% sequence identity to at least 18 nucleotides of a sequence selected from the group consisting of SEQ ID NOs: 1-43 and 87-118 (preferably selected from the group consisting of SEQ ID NOs: 1-41 and 87-118). In some embodiments, the guide sequences comprise or consist of a sequence having 100% sequence identity to at least 18 contiguous nucleotides of a sequence selected from the group consisting of SEQ ID NOs: 1-43 and 87-118 (preferably selected from the group consisting of SEQ ID NOs: 1-41 and 87-118). In some embodiments, the guide sequences comprise or consist of a sequence having 100% sequence identity to at least 19 nucleotides of a sequence selected from the group consisting of SEQ ID NOs: 1-43 and 87-118 (preferably selected from the group consisting of SEQ ID NOs: 1-41 and 87-118). In some embodiments, the guide sequences comprise or consist of a sequence selected from the group consisting of SEQ ID NOs: 1-43 and 87-118 (preferably selected from the group consisting of SEQ ID NOs: 1-41 and 87-118).

### Guide RNAs

In some embodiments, the present invention is directed to gRNAs that comprise or consist of guide sequences as set forth in the “Guide Sequences” section above. In some embodiments, the gRNAs comprise or consist of a guide sequence, as set forth in the “Guide Sequences” section above, and a repeat sequence that base-pairs with a portion of a given tracrRNA sequence. Those skilled in the art may readily select or design a tracrRNA sequence to be used in conjunction with a given guide sequence and a given Cas protein using methods in the art. In some embodiments, the gRNAs comprise or consists of a guide sequence, as set forth in the “Guide Sequences” section above, a repeat sequence that base-pairs with a portion of a given tracrRNA sequence, and the given tracrRNA sequence. In some embodiments, the gRNAs are two-component gRNAs in which the guide sequence and the tracrRNA sequence are provided as separate nucleotide molecules (duplex crRNA:tracrRNA). In some embodiments, the tracrRNA sequence comprises or consists of:

(SEQ ID NO: 151)
UAGCAAGUUAAAAUAAGGCUAGUCCGUUAUCAACUUGAAAAAGUGGCACC
GAGUCGGUGCUUUU

and the crRNA repeat sequence that base-pairs with the tracrRNA sequence comprises or consists of:

(SEQ ID NO: 152)
GUUUUAGAGCUA

In some embodiments, the gRNAs are single guide RNAs (sgRNAs) in which the guide sequence and the tracrRNA sequence are combined into a single nucleotide molecule. In some embodiments, the sgRNAs comprise a crRNA repeat sequence fused to a tracrRNA sequence (crRNA repeat-tracrRNA fusion). In some embodiments, the crRNA re eat-tracrRNA fusion comprises or consists of:

(SEQ ID NO: 153)
GUUUUAGAGCUAGAAAUAGCAAGUUAAAAUAAGGCUAGUCCGUUAUCAAC
UUGAAAAAGUGGCACCGAGUCGGUGCUUUU

### Encoding Nucleotide Molecules

In some embodiments, the present invention is directed to nucleotide molecules that encode the gRNAs as set forth in the “Guide RNAs” section above. In some embodiments, such “encoding nucleotide molecules” are provided as expression cassettes and/or as parts of expression vectors or plasmids. Suitable expression cassettes and expression vectors may be readily selected or designed using methods in the art. In some embodiments, the expression vector is an adeno-associated virus (AAV) vector or plasmid. Exemplary encoding nucleotide molecules comprise at least 17, at least 18, or at least 19 nucleotides of the non-complementary strand of the target sequence of the given guide sequence. For example, an encoding nucleotide molecule that encodes a gRNA having SEQ ID NO: 1 as the guide sequence comprises SEQ ID NO: 44 and, at least, a sequence that encodes the repeat sequence that base-pairs with the portion of a given tracrRNA sequence. The phrase “encoding nucleotide molecule(s) thereof” when used in conjunction with one or more given gRNAs means one or more nucleotide molecules that encode the one or more gRNAs. When a gRNA is two-component gRNA (a duplex crRNA:tracrRNA), the “encoding nucleotide molecule(s) thereof” may be (a) a single nucleic acid molecule that encodes both the guide sequence and the tracrRNA sequence, or (b) two separate nucleic acid molecules-one encoding the guide sequence and the other encoding the tracrRNA sequence. In embodiments where a first gRNA and a second gRNA are provided, one or both may be a two-component gRNA. When the phrase “encoding nucleotide molecule(s) thereof” is used in conjunction with first and second gRNAs—for example, “a first gRNA and a second gRNA or encoding nucleotide molecule(s) thereof”—the “encoding nucleotide molecule(s) thereof” may be (a) a single nucleic acid molecule that encodes both the guide sequences and the tracrRNA sequences of both the first and second gRNAs, or (b) multiple nucleic acid molecules, e.g., one encoding the guide sequence and tracrRNA sequence of the first gRNA and another encoding the guide sequence and tracrRNA sequence of the second gRNA, three nucleic acid molecules—a first nucleic acid molecule encoding the guide sequences and the tracrRNA sequences of the first gRNA and the second and third nucleic acid molecules encoding the guide sequence and the tracrRNA sequence of the second gRNA, etc. Additionally, the phrase “a first gRNA and a second gRNA or encoding nucleotide molecule(s) thereof” does not require that encoding nucleotides for both the first gRNA and the second gRNA are provided or employed. That is, encoding nucleotide molecule(s) of only one gRNA may be provided. For example, a composition comprising “a first gRNA and a second gRNA or encoding nucleotide molecule(s) thereof” may comprise (a) the first gRNA and the second gRNA, (b) a first gRNA and one or more nucleotide molecule(s) that encode the second gRNA, or vice versa, or (c) one or more nucleotide molecule(s) that encode the first gRNA and one or more nucleotide molecule(s) that encode the second gRNA.

### Compositions

In some embodiments, the present invention is directed to compositions comprising one or more of guide sequences and/or one or more gRNAs (or encoding nucleotide molecule(s) thereof) as set forth in the “Guide Sequences”, “Guide RNAs”, and “Encoding Nucleotide Molecules” sections above. In some embodiments, the compositions further include one or more gRNAs that comprise guide sequences having 100% sequence identity to at least 17 nucleotides of SEQ ID NO: 42 or SEQ ID NO: 43, or one or more encoding nucleotide molecule(s) thereof. In some embodiments, the compositions further include one or more gRNAs that comprise guide sequences having 100% sequence identity to at least 18 nucleotides of SEQ ID NO: 42 or SEQ ID NO: 43, or one or more encoding nucleotide molecule(s) thereof. In some embodiments, the compositions further include one or more gRNAs that comprise guide sequences having 100% sequence identity to at least 19 nucleotides of SEQ ID NO: 42 or SEQ ID NO: 43, or one or more encoding nucleotide molecule(s) thereof. In some embodiments, the compositions further include one or more gRNAs that comprise guide sequences having SEQ ID NO: 42 or SEQ ID NO: 43, or one or more encoding nucleotide molecule(s) thereof.

In some embodiments, the compositions comprise a first gRNA having a first guide sequence and a second gRNA having a second guide sequence (or encoding nucleotide molecule(s) thereof). In some embodiments, the first guide sequence comprises or consists of a sequence that has 100% sequence identity to at least 17, at least 18, at least 19, or all 20 nucleotides of SEQ ID NO: 1 or SEQ ID NO: 2 and the second guide sequence comprises or consists of a sequence that has 100% sequence identity to at least 17, at least 18, at least 19, or all 20 nucleotides of SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 39, SEQ ID NO: 40, SEQ ID NO: 41, or SEQ ID NO: 43. In some embodiments, the first guide sequence comprises or consists of a sequence that has 100% sequence identity to at least 17, at least 18, at least 19, or all 20 nucleotides of SEQ ID NO: 1 and the second guide sequence comprises or consists of a sequence that has 100% sequence identity to at least 17, at least 18, at least 19, or all 20 nucleotides of SEQ ID NO: 8, SEQ ID NO: 16, SEQ ID NO: 19, SEQ ID NO: 27, SEQ ID NO: 35, SEQ ID NO: 39, or SEQ ID NO: 43. In some embodiments, the first guide sequence comprises or consists of SEQ ID NO: 1 and the second guide sequence comprises or consists of SEQ ID NO: 8, SEQ ID NO: 16, SEQ ID NO: 19, SEQ ID NO: 27, SEQ ID NO: 35, SEQ ID NO: 39, or SEQ ID NO: 43.

In some embodiments, the first guide sequence comprises or consists of a sequence that has 100% sequence identity to at least 17, at least 18, at least 19, or all 20 nucleotides of SEQ ID NO: 42 and the second guide sequence comprises or consists of a sequence that has 100% sequence identity to at least 17, at least 18, at least 19, or all 20 nucleotides of SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 39, SEQ ID NO: 40, or SEQ ID NO: 41. In some embodiments, the first guide sequence comprises or consists of a sequence that has 100% sequence identity to at least 17, at least 18, at least 19, or all 20 nucleotides of SEQ ID NO: 42 and the second guide sequence comprises or consists of a sequence that has 100% sequence identity to at least 17, at least 18, at least 19, or all 20 nucleotides of SEQ ID NO: 8, SEQ ID NO: 16, SEQ ID NO: 19, SEQ ID NO: 27, SEQ ID NO: 35, or SEQ ID NO: 39. In some embodiments, the first guide sequence comprises or consists of a sequence that has 100% sequence identity to at least 17, at least 18, at least 19, or all 20 nucleotides of SEQ ID NO: 42 and the second guide sequence comprises or consists of a sequence that has 100% sequence identity to at least 17, at least 18, at least 19, or all 20 nucleotides of SEQ ID NO: 19. In some embodiments, the first guide sequence comprises or consists of a sequence that has 100% sequence identity to at least 17, at least 18, at least 19, or all 20 nucleotides of SEQ ID NO: 42 and the second guide sequence comprises or consists of a sequence that has 100% sequence identity to at least 17, at least 18, at least 19, or all 20 nucleotides of SEQ ID NO: 35. In some embodiments, the first guide sequence comprises or consists of SEQ ID NO: 42 and the second guide sequence comprises or consists of SEQ ID NO: 19. In some embodiments, the first guide sequence comprises or consists of SEQ ID NO: 42 and the second guide sequence comprises or consists of SEQ ID NO: 35.

In some embodiments, the first guide sequence comprises or consists of a sequence that has 100% sequence identity to at least 17, at least 18, at least 19, or all 20 nucleotides of SEQ ID NO: 36, SEQ ID NO: 37, or SEQ ID NO: 38 and the second guide sequence comprises or consists of a sequence that has 100% sequence identity to at least 17, at least 18, at least 19, or all 20 nucleotides of SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 39, SEQ ID NO: 40, SEQ ID NO: 41, or SEQ ID NO: 43. In some embodiments, the first guide sequence comprises or consists of a sequence that has 100% sequence identity to at least 17, at least 18, at least 19, or all 20 nucleotides of SEQ ID NO: 36, SEQ ID NO: 37, or SEQ ID NO: 38 and the second guide sequence comprises or consists of a sequence that has 100% sequence identity to at least 17, at least 18, at least 19, or all 20 nucleotides of SEQ ID NO: 8, SEQ ID NO: 16, SEQ ID NO: 19, SEQ ID NO: 27, SEQ ID NO: 35, SEQ ID NO: 39, or SEQ ID NO: 43. In some embodiments, the first guide sequence comprises or consists of a sequence that has 100% sequence identity to at least 17, at least 18, at least 19, or all 20 nucleotides of SEQ ID NO: 37 and the second guide sequence comprises or consists of a sequence that has 100% sequence identity to at least 17, at least 18, at least 19, or all 20 nucleotides of SEQ ID NO: 16. In some embodiments, the first guide sequence comprises or consists of a sequence that has 100% sequence identity to at least 17, at least 18, at least 19, or all 20 nucleotides of SEQ ID NO: 37 and the second guide sequence comprises or consists of a sequence that has 100% sequence identity to at least 17, at least 18, at least 19, or all 20 nucleotides of SEQ ID NO: 27. In some embodiments, the first guide sequence comprises or consists of a sequence that has 100% sequence identity to at least 17, at least 18, at least 19, or all 20 nucleotides of SEQ ID NO: 37 and the second guide sequence comprises or consists of a sequence that has 100% sequence identity to at least 17, at least 18, at least 19, or all 20 nucleotides of SEQ ID NO: 39. In some embodiments, the first guide sequence comprises or consists of SEQ ID NO: 37 and the second guide sequence comprises or consists of SEQ ID NO: 16. In some embodiments, the first guide sequence comprises or consists of SEQ ID NO: 37 and the second guide sequence comprises or consists of SEQ ID NO: 27. In some embodiments, the first guide sequence comprises or consists of SEQ ID NO: 37 and the second guide sequence comprises or consists of SEQ ID NO: 39.

In some embodiments, the first guide sequence comprises or consists of a sequence that has 100% sequence identity to at least 17, at least 18, at least 19, or all 20 nucleotides of SEQ ID NO: 5 or SEQ ID NO: 6 and the second guide sequence comprises or consists of a sequence that has 100% sequence identity to at least 17, at least 18, at least 19, or all 20 nucleotides of SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 39, SEQ ID NO: 40, SEQ ID NO: 41, or SEQ ID NO: 43. In some embodiments, the first guide sequence comprises or consists of a sequence that has 100% sequence identity to at least 17, at least 18, at least 19, or all 20 nucleotides of SEQ ID NO: 5 or SEQ ID NO: 6 and the second guide sequence comprises or consists of a sequence that has 100% sequence identity to at least 17, at least 18, at least 19, or all 20 nucleotides of SEQ ID NO: 8, SEQ ID NO: 16, SEQ ID NO: 19, SEQ ID NO: 27, SEQ ID NO: 35, SEQ ID NO: 39, or SEQ ID NO: 43. In some embodiments, the first guide sequence comprises or consists of a sequence that has 100% sequence identity to at least 17, at least 18, at least 19, or all 20 nucleotides of SEQ ID NO: 5 and the second guide sequence comprises or consists of a sequence that has 100% sequence identity to at least 17, at least 18, at least 19, or all 20 nucleotides of SEQ ID NO: 8. In some embodiments, the first guide sequence comprises or consists of SEQ ID NO: 5 and the second guide sequence comprises or consists of SEQ ID NO: 8.

In some embodiments, the first guide sequence comprises or consists of a sequence that has 100% sequence identity to at least 17, at least 18, at least 19, or all 20 nucleotides of SEQ ID NO: 5, SEQ ID NO: 6, SEQ ID NO: 88, SEQ ID NO: 89, SEQ ID NO: 90, SEQ ID NO: 91, SEQ ID NO: 92, SEQ ID NO: 93, SEQ ID NO: 94, or SEQ ID NO: 95 and the second guide sequence comprises or consists of a sequence that has 100% sequence identity to at least 17, at least 18, at least 19, or all 20 nucleotides of SEQ ID NO: 7, SEQ ID NO: 8, SEQ ID NO: 9, SEQ ID NO: 96, SEQ ID NO: 97, or SEQ ID NO: 98.

In some embodiments, the first guide sequence comprises or consists of a sequence that has 100% sequence identity to at least 17, at least 18, at least 19, or all 20 nucleotides of SEQ ID NO: 36, SEQ ID NO: 37, SEQ ID NO: 38, SEQ ID NO: 111, SEQ ID NO: 112, SEQ ID NO: 113, SEQ ID NO: 114, or SEQ ID NO: 115 and the second guide sequence comprises or consists of a sequence that has 100% sequence identity to at least 17, at least 18, at least 19, or all 20 nucleotides of SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 99, SEQ ID NO: 100, SEQ ID NO: 101, SEQ ID NO: 102, or SEQ ID NO: 103.

In some embodiments, the first guide sequence comprises or consists of a sequence that has 100% sequence identity to at least 17, at least 18, at least 19, or all 20 nucleotides of SEQ ID NO: 42 and the second guide sequence comprises or consists of a sequence that has 100% sequence identity to at least 17, at least 18, at least 19, or all 20 nucleotides of SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 99, SEQ ID NO: 100, SEQ ID NO: 101, SEQ ID NO: 102, or SEQ ID NO: 103.

In some embodiments, the first guide sequence comprises or consists of a sequence that has 100% sequence identity to at least 17, at least 18, at least 19, or all 20 nucleotides of SEQ ID NO: 36, SEQ ID NO: 37, SEQ ID NO: 38, SEQ ID NO: 111, SEQ ID NO: 112, SEQ ID NO: 113, SEQ ID NO: 114, or SEQ ID NO: 115 and the second guide sequence comprises or consists of a sequence that has 100% sequence identity to at least 17, at least 18, at least 19, or all 20 nucleotides of SEQ ID NO: 22, SEQ ID NO: 23, SEQ ID NO: 24, SEQ ID NO: 25, SEQ ID NO: 26, SEQ ID NO: 27, SEQ ID NO: 106, or SEQ ID NO: 107.

In some embodiments, the first guide sequence comprises or consists of a sequence that has 100% sequence identity to at least 17, at least 18, at least 19, or all 20 nucleotides of SEQ ID NO: 42 and the second guide sequence comprises or consists of a sequence that has 100% sequence identity to at least 17, at least 18, at least 19, or all 20 nucleotides of SEQ ID NO: 22, SEQ ID NO: 23, SEQ ID NO: 24, SEQ ID NO: 25, SEQ ID NO: 26, SEQ ID NO: 27, SEQ ID NO: 106, or SEQ ID NO: 107.

In some embodiments, the first guide sequence comprises or consists of a sequence that has 100% sequence identity to at least 17, at least 18, at least 19, or all 20 nucleotides of SEQ ID NO: 36, SEQ ID NO: 37, SEQ ID NO: 38, SEQ ID NO: 111, SEQ ID NO: 112, SEQ ID NO: 113, SEQ ID NO: 114, or SEQ ID NO: 115 and the second guide sequence comprises or consists of a sequence that has 100% sequence identity to at least 17, at least 18, at least 19, or all 20 nucleotides of SEQ ID NO: 39, SEQ ID NO: 40, SEQ ID NO: 41, SEQ ID NO: 116, SEQ ID NO: 117, or SEQ ID NO: 118.

In some embodiments, the first guide sequence comprises or consists of a sequence that has 100% sequence identity to at least 17, at least 18, at least 19, or all 20 nucleotides of SEQ ID NO: 42 and the second guide sequence comprises or consists of a sequence that has 100% sequence identity to at least 17, at least 18, at least 19, or all 20 nucleotides of SEQ ID NO: 43.

In some embodiments, the first guide sequence comprises or consists of a sequence that has 100% sequence identity to at least 17, at least 18, at least 19, or all 20 nucleotides of SEQ ID NO: 42 and the second guide sequence comprises or consists of a sequence that has 100% sequence identity to at least 17, at least 18, at least 19, or all 20 nucleotides of SEQ ID NO: 3, or SEQ ID NO: 4.

In some embodiments, the first guide sequence comprises or consists of a sequence that has 100% sequence identity to at least 17, at least 18, at least 19, or all 20 nucleotides of SEQ ID NO: 42 and the second guide sequence comprises or consists of a sequence that has 100% sequence identity to at least 17, at least 18, at least 19, or all 20 nucleotides of SEQ ID NO: 7, SEQ ID NO: 8, SEQ ID NO: 9, SEQ ID NO: 96, SEQ ID NO: 97, or SEQ ID NO: 98.

In some embodiments, the first guide sequence comprises or consists of a sequence that has 100% sequence identity to at least 17, at least 18, at least 19, or all 20 nucleotides of SEQ ID NO: 7, SEQ ID NO: 8, SEQ ID NO: 9, SEQ ID NO: 96, SEQ ID NO: 97, or SEQ ID NO: 98 and the second guide sequence comprises or consists of a sequence that has 100% sequence identity to at least 17, at least 18, at least 19, or all 20 nucleotides of SEQ ID NO: 17, SEQ ID NO: 18, SEQ ID NO: 19, SEQ ID NO: 20, SEQ ID NO: 21, SEQ ID NO: 104, or SEQ ID NO: 105.

In some embodiments, the first guide sequence comprises or consists of a sequence that has 100% sequence identity to at least 17, at least 18, at least 19, or all 20 nucleotides of SEQ ID NO: 7, SEQ ID NO: 8, SEQ ID NO: 9, SEQ ID NO: 96, SEQ ID NO: 97, or SEQ ID NO: 98 and the second guide sequence comprises or consists of a sequence that has 100% sequence identity to at least 17, at least 18, at least 19, or all 20 nucleotides of SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 108, SEQ ID NO: 109, or SEQ ID NO: 110.

In some embodiments, the first guide sequence comprises or consists of a sequence that has 100% sequence identity to at least 17, at least 18, at least 19, or all 20 nucleotides of SEQ ID NO: 7, SEQ ID NO: 8, SEQ ID NO: 9, SEQ ID NO: 96, SEQ ID NO: 97, or SEQ ID NO: 98 and the second guide sequence comprises or consists of a sequence that has 100% sequence identity to at least 17, at least 18, at least 19, or all 20 nucleotides of SEQ ID NO: 43.

In some embodiments, the first guide sequence comprises or consists of a sequence that has 100% sequence identity to at least 17, at least 18, at least 19, or all 20 nucleotides of SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 99, SEQ ID NO: 100, SEQ ID NO: 101, SEQ ID NO: 102, or SEQ ID NO: 103 and the second guide sequence comprises or consists of a sequence that has 100% sequence identity to at least 17, at least 18, at least 19, or all 20 nucleotides of SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 108, SEQ ID NO: 109, or SEQ ID NO: 110.

In some embodiments, the first guide sequence comprises or consists of a sequence that has 100% sequence identity to at least 17, at least 18, at least 19, or all 20 nucleotides of SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 99, SEQ ID NO: 100, SEQ ID NO: 101, SEQ ID NO: 102, or SEQ ID NO: 103 and the second guide sequence comprises or consists of a sequence that has 100% sequence identity to at least 17, at least 18, at least 19, or all 20 nucleotides of SEQ ID NO: 43.

In some embodiments, the first guide sequence comprises or consists of a sequence that has 100% sequence identity to at least 17, at least 18, at least 19, or all 20 nucleotides of SEQ ID NO: 22, SEQ ID NO: 23, SEQ ID NO: 24, SEQ ID NO: 25, SEQ ID NO: 26, SEQ ID NO: 27, SEQ ID NO: 106, or SEQ ID NO: 107 and the second guide sequence comprises or consists of a sequence that has 100% sequence identity to at least 17, at least 18, at least 19, or all 20 nucleotides of SEQ ID NO: 43.

In some embodiments, the first guide sequence comprises or consists of a sequence that has 100% sequence identity to at least 17, at least 18, at least 19, or all 20 nucleotides of SEQ ID NO: 1, SEQ ID NO: 2, or SEQ ID NO: 87 and the second guide sequence comprises or consists of a sequence that has 100% sequence identity to at least 17, at least 18, at least 19, or all 20 nucleotides of a sequence selected from the group consisting of SEQ ID NOs: 5-35, 39-41, 43, 88-110, and 116-118.

In some embodiments, the first guide sequence comprises or consists of a sequence that has 100% sequence identity to at least 17, at least 18, at least 19, or all 20 nucleotides of SEQ ID NO: 42 and the second guide sequence comprises or consists of a sequence that has 100% sequence identity to at least 17, at least 18, at least 19, or all 20 nucleotides of a sequence selected from the group consisting of SEQ ID NOs: 3-35, 39-41, 43, 88-110, and 116-118.

In some embodiments, the first guide sequence comprises or consists of a sequence that has 100% sequence identity to at least 17, at least 18, at least 19, or all 20 nucleotides of SEQ ID NO: 36, SEQ ID NO: 37, SEQ ID NO: 38, SEQ ID NO: 111, SEQ ID NO: 112, SEQ ID NO: 113, SEQ ID NO: 114, or SEQ ID NO: 115 and the second guide sequence comprises or consists of a sequence that has 100% sequence identity to at least 17, at least 18, at least 19, or all 20 nucleotides of a sequence selected from the group consisting of SEQ ID NOs: 3-35, 39-41, 43, 88-110, and 116-118.

In some embodiments, the first guide sequence comprises or consists of a sequence that has 100% sequence identity to at least 17, at least 18, at least 19, or all 20 nucleotides of SEQ ID NO: 5, SEQ ID NO: 6, SEQ ID NO: 88, SEQ ID NO: 89, SEQ ID NO: 90, SEQ ID NO: 91, SEQ ID NO: 92, SEQ ID NO: 93, SEQ ID NO: 94, or SEQ ID NO: 95 and the second guide sequence comprises or consists of a sequence that has 100% sequence identity to at least 17, at least 18, at least 19, or all 20 nucleotides of a sequence selected from the group consisting of SEQ ID NOs: 7-35, 39-41, 43, 96-110, and 116-118.

In some embodiments, the second guide sequence comprises or consists of a sequence that has 100% sequence identity to at least 17, at least 18, at least 19, or all 20 nucleotides of SEQ ID NO: 43 and the first guide sequence comprises or consists of a sequence that has 100% sequence identity to at least 17, at least 18, at least 19, or all 20 nucleotides of a sequence selected from the group consisting of SEQ ID NOs: 1-38, 42, and 87-115.

In some embodiments, the second guide sequence comprises or consists of a sequence that has 100% sequence identity to at least 17, at least 18, at least 19, or all 20 nucleotides of SEQ ID NO: 39, SEQ ID NO: 40, SEQ ID NO: 41, SEQ ID NO: 116, SEQ ID NO: 117, or SEQ ID NO: 118 and the first guide sequence comprises or consists of a sequence that has 100% sequence identity to at least 17, at least 18, at least 19, or all 20 nucleotides of a sequence selected from the group consisting of SEQ ID NOs: 1-38, 42, and 87-115.

In some embodiments, the second guide sequence comprises or consists of a sequence that has 100% sequence identity to at least 17, at least 18, at least 19, or all 20 nucleotides of SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 108, SEQ ID NO: 109, or SEQ ID NO: 110 and the first guide sequence comprises or consists of a sequence that has 100% sequence identity to at least 17, at least 18, at least 19, or all 20 nucleotides of a sequence selected from the group consisting of SEQ ID NOs: 1-21, 36-38, 42, 87-105, and 111-115.

In some embodiments, the second guide sequence comprises or consists of a sequence that has 100% sequence identity to at least 17, at least 18, at least 19, or all 20 nucleotides of SEQ ID NO: 22, SEQ ID NO: 23, SEQ ID NO: 24, SEQ ID NO: 25, SEQ ID NO: 26, SEQ ID NO: 27, SEQ ID NO: 106, or SEQ ID NO: 107 and the first guide sequence comprises or consists of a sequence that has 100% sequence identity to at least 17, at least 18, at least 19, or all 20 nucleotides of a sequence selected from the group consisting of SEQ ID NOs: 1-21, 36-38, 42, 87-105, and 111-115.

In some embodiments, the second guide sequence comprises or consists of a sequence that has 100% sequence identity to at least 17, at least 18, at least 19, or all 20 nucleotides of SEQ ID NO: 17, SEQ ID NO: 18, SEQ ID NO: 19, SEQ ID NO: 20, SEQ ID NO: 21, SEQ ID NO: 104, or SEQ ID NO: 105 and the first guide sequence comprises or consists of a sequence that has 100% sequence identity to at least 17, at least 18, at least 19, or all 20 nucleotides of a sequence selected from the group consisting of SEQ ID NOs: 1-9, 36-38, 42, 87-98, and 111-115.

In some embodiments, the second guide sequence comprises or consists of a sequence that has 100% sequence identity to at least 17, at least 18, at least 19, or all 20 nucleotides of SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 99, SEQ ID NO: 100, SEQ ID NO: 101, SEQ ID NO: 102, or SEQ ID NO: 103 and the first guide sequence comprises or consists of a sequence that has 100% sequence identity to at least 17, at least 18, at least 19, or all 20 nucleotides of a sequence selected from the group consisting of SEQ ID NOs: 1-9, 36-38, 42, 87-98, and 111-115.

In some embodiments, the second guide sequence comprises or consists of a sequence that has 100% sequence identity to at least 17, at least 18, at least 19, or all 20 nucleotides of SEQ ID NO: 7, SEQ ID NO: 8, SEQ ID NO: 9, SEQ ID NO: 96, SEQ ID NO: 97, or SEQ ID NO: 98 and the first guide sequence comprises or consists of a sequence that has 100% sequence identity to at least 17, at least 18, at least 19, or all 20 nucleotides of a sequence selected from the group consisting of SEQ ID NOs: 1-6, 36-38, 42, 87-95, and 111-115.

In some embodiments, the compositions further include a Cas protein (or a nucleic acid molecule encoding the Cas protein). In some embodiments, the compositions further include a buffer, a diluent, a carrier, a reconstitution solution, and/or a wash buffer, etc.

### Kits

In some embodiments, the present invention is directed to kits comprising one or more guide sequences, as set forth in the “Guide Sequences” section above, packaged together. In some embodiments, the present invention is directed to kits comprising one or more gRNAs (or encoding nucleotide molecule(s) thereof), as set forth in the “Guide RNAs” and “Encoding Nucleotide Molecules” sections above, packaged together. The “Guide Sequences” and “Guide RNAs” may be provided in the form of a composition, e.g., a composition as set forth in the “Composition” section above. In some embodiments, the kits further comprise one or more Cas proteins (or one or more nucleic acid molecules encoding the one or more Cas proteins). In some embodiments, the kits further comprise one or more devices and/or vectors for delivering one or more gRNAs (or encoding nucleotide molecule(s) thereof) to a subject or cell.

In some embodiments, the kits further include one or more additional reagents, selected from: dilution buffers; reconstitution solutions; wash buffers; control reagents; control expression vectors or RNA polynucleotides; reagents for production of a Cas protein from DNA or RNA; reagents for production of a gRNA from DNA; and the like. In some embodiments, the kits further include instructions for use.

### Methods

In some embodiments, the present invention provides methods for modifying a dystrophin gene in a cell or in a subject, which comprises introducing into the cell or the subject: (a) a Cas protein or a nucleotide sequence encoding the Cas protein; and (b1) a single gRNA (or encoding nucleotide molecule(s) thereof), or (b2) a first gRNA having a first guide sequence and a second gRNA having a second guide sequence (or encoding nucleotide molecule(s) thereof), wherein the first guide sequence and/or the second guide sequence is as provided in the “Guide Sequences” section above. In some embodiments, the Cas protein or the nucleotide sequence encoding the Cas protein, the single gRNA, the first gRNA, the second gRNA, and/or one or more encoding nucleotide molecule(s) (of the single gRNA, first gRNA and/or second gRNA) are provided in the form of a composition or kit. In some embodiments, one or more of the “Guide RNAs”, “Encoding Nucleotide Molecules”, “Compositions”, and/or “Kits” as disclosed above are used. In some embodiments, the dystrophin gene is a human dystrophin gene (e.g., Gene ID 1756, Accession No. NG_012232.1). In some embodiments, the dystrophin gene is a mutant dystrophin gene, e.g., a human dystrophin gene having one or more mutations.

In some embodiments, the method results in a cleavage in or right before exon 44 of the dystrophin gene in the cell or the subject. In some embodiments, the single gRNA guide sequence employed comprises a sequence having 100% sequence identity to at least 17, at least 18, at least 19, or all 20 nucleotides of SEQ ID NO: 1 or SEQ ID NO: 2. In some embodiments, the single gRNA guide sequence comprises SEQ ID NO: 1.

In some embodiments, the method results in a cleavage in or right before exon 46 of the dystrophin gene in the cell or the subject. In some embodiments, the single gRNA guide sequence employed comprises a sequence having 100% sequence identity to at least 17, at least 18, at least 19, or all 20 nucleotides of SEQ ID NO: 3 or SEQ ID NO: 4. In some embodiments, the single gRNA guide sequence comprises SEQ ID NO: 4.

In some embodiments, the method results in cleavages in exon 44 and intron 46 of the dystrophin gene in the cell or the subject. In some embodiments, the method results in cleavages in exon 44 and intron 45 of the dystrophin gene in the cell or the subject. In some embodiments, the method results in cleavages in exon 44 and intron 48 of the dystrophin gene in the cell or the subject. In some embodiments, the method results in cleavages in exon 44 and intron 51 of the dystrophin gene in the cell or the subject. In some embodiments, the method results in cleavages in exon 44 and intron 55 of the dystrophin gene in the cell or the subject. In some embodiments, the method results in cleavages in exon 46 and intron 46 of the dystrophin gene in the cell or the subject. In some embodiments, the method results in cleavages in exon 46 and intron 48 of the dystrophin gene in the cell or the subject. In some embodiments, the method results in cleavages in exon 46 and intron 51 of the dystrophin gene in the cell or the subject. In some embodiments, the method results in cleavages in exon 46 and intron 55 of the dystrophin gene in the cell or the subject. In some embodiments, the method results in cleavages in intron 44 and intron 46 of the dystrophin gene in the cell or the subject. In some embodiments, the method results in cleavages in intron 45 and intron 46 of the dystrophin gene in the cell or the subject. In some embodiments, the method results in cleavages in intron 44 and intron 48 of the dystrophin gene in the cell or the subject. In some embodiments, the method results in cleavages in intron 44 and intron 51 of the dystrophin gene in the cell or the subject. In some embodiments, the method results in cleavages in intron 44 and intron 55 of the dystrophin gene in the cell or the subject. In some embodiments, the method results in cleavages in intron 45 and intron 46 of the dystrophin gene in the cell or the subject. In some embodiments, the method results in cleavages in intron 45 and intron 48 of the dystrophin gene in the cell or the subject. In some embodiments, the method results in cleavages in intron 45 and intron 51 of the dystrophin gene in the cell or the subject. In some embodiments, the method results in cleavages in intron 45 and intron 55 of the dystrophin gene in the cell or the subject. In some embodiments, the method results in cleavages in intron 46 and 48, intron 46 and intron 51, intron 46 and intron 55, intron 48 and intron 51, intron 48 and intron 55, or intron 51 and intron 55 of the dystrophin gene in the cell or the subject.

In some embodiments, the method results in a deletion, frameshift, or splice site disruption of exon 44 of the dystrophin gene in the cell or the subject. In some embodiments, the method results in a deletion, frameshift, or splice site disruption of exon 46 of the dystrophin gene in the cell or the subject. In some embodiments, the method results in a deletion of exon 44 to exon 46 of the dystrophin gene in the cell or the subject. In some embodiments, the method results in a deletion of exon 44 to exon 45 of the dystrophin gene in the cell or the subject. In some embodiments, the method results in a deletion of exon 44 to exon 48 of the dystrophin gene in the cell or the subject. In some embodiments, the method results in a deletion of exon 44 to exon 51 of the dystrophin gene in the cell or the subject. In some embodiments, the method results in a deletion of exon 44 to exon 55 of the dystrophin gene in the cell or the subject. In some embodiments, the method results in a deletion of exon 46 of the dystrophin gene in the cell or the subject. In some embodiments, the method results in a deletion of exon 46 to exon 48 of the dystrophin gene in the cell or the subject. In some embodiments, the method results in a deletion of exon 46 to exon 51 of the dystrophin gene in the cell or the subject. In some embodiments, the method results in a deletion of exon 46 to exon 55 of the dystrophin gene in the cell or the subject. In some embodiments, the method results in a deletion of exon 45 to exon 46 of the dystrophin gene in the cell or the subject. In some embodiments, the method results in a deletion of exon 45 to exon 48 of the dystrophin gene in the cell or the subject. In some embodiments, the method results in a deletion of exon 45 to exon 51 of the dystrophin gene in the cell or the subject. In some embodiments, the method results in a deletion of exon 45 to exon 55 of the dystrophin gene in the cell or the subject. In some embodiments, the method results in a deletion of exon 46 of the dystrophin gene in the cell or the subject. In some embodiments, the method results in a deletion of exon 46 to exon 48 of the dystrophin gene in the cell or the subject. In some embodiments, the method results in a deletion of exon 46 to exon 51 of the dystrophin gene in the cell or the subject. In some embodiments, the method results in a deletion of exon 46 to exon 55 of the dystrophin gene in the cell or the subject. In some embodiments, the method results in a deletion of exons 47-48, 47-51, 47-55, 49-51, 49-55, or 52-55 of the dystrophin gene in the cell or the subject

In some embodiments, the first guide sequence employed comprises or consists of a sequence that has 100% sequence identity to at least 17, at least 18, at least 19, or all 20 nucleotides of SEQ ID NO: 1 or SEQ ID NO: 2 and the second guide sequence employed comprises or consists of a sequence that has 100% sequence identity to at least 17, at least 18, at least 19, or all 20 nucleotides of SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 39, SEQ ID NO: 40, SEQ ID NO: 41, or SEQ ID NO: 43. In some embodiments, the first guide sequence employed comprises or consists of a sequence that has 100% sequence identity to at least 17, at least 18, at least 19, or all 20 nucleotides of SEQ ID NO: 1 and the second guide sequence employed comprises or consists of a sequence that has 100% sequence identity to at least 17, at least 18, at least 19, or all 20 nucleotides of SEQ ID NO: 8, SEQ ID NO: 16, SEQ ID NO: 19, SEQ ID NO: 27, SEQ ID NO: 35, SEQ ID NO: 39, or SEQ ID NO: 43. In some embodiments, the first guide sequence employed comprises or consists of SEQ ID NO: 1 and the second guide sequence employed comprises or consists of SEQ ID NO: 8, SEQ ID NO: 16, SEQ ID NO: 19, SEQ ID NO: 27, SEQ ID NO: 35, SEQ ID NO: 39, or SEQ ID NO: 43.

In some embodiments, the first guide sequence employed comprises or consists of a sequence that has 100% sequence identity to at least 17, at least 18, at least 19, or all 20 nucleotides of SEQ ID NO: 42 and the second guide sequence employed comprises or consists of a sequence that has 100% sequence identity to at least 17, at least 18, at least 19, or all 20 nucleotides of SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 39, SEQ ID NO: 40, or SEQ ID NO: 41. In some embodiments, the first guide sequence employed comprises or consists of a sequence that has 100% sequence identity to at least 17, at least 18, at least 19, or all 20 nucleotides of SEQ ID NO: 42 and the second guide sequence employed comprises or consists of a sequence that has 100% sequence identity to at least 17, at least 18, at least 19, or all 20 nucleotides of SEQ ID NO: 8, SEQ ID NO: 16, SEQ ID NO: 19, SEQ ID NO: 27, SEQ ID NO: 35, or SEQ ID NO: 39. In some embodiments, the first guide sequence employed comprises or consists of a sequence that has 100% sequence identity to at least 17, at least 18, at least 19, or all 20 nucleotides of SEQ ID NO: 42 and the second guide sequence employed comprises or consists of a sequence that has 100% sequence identity to at least 17, at least 18, at least 19, or all 20 nucleotides of SEQ ID NO: 19. In some embodiments, the first guide sequence employed comprises or consists of a sequence that has 100% sequence identity to at least 17, at least 18, at least 19, or all 20 nucleotides of SEQ ID NO: 42 and the second guide sequence employed comprises or consists of a sequence that has 100% sequence identity to at least 17, at least 18, at least 19, or all 20 nucleotides of SEQ ID NO: 35. In some embodiments, the first guide sequence employed comprises or consists of SEQ ID NO: 42 and the second guide sequence employed comprises or consists of SEQ ID NO: 19. In some embodiments, the first guide sequence employed comprises or consists of SEQ ID NO: 42 and the second guide sequence employed comprises or consists of SEQ ID NO: 35.

In some embodiments, the first guide sequence employed comprises or consists of a sequence that has 100% sequence identity to at least 17, at least 18, at least 19, or all 20 nucleotides of SEQ ID NO: 36, SEQ ID NO: 37, or SEQ ID NO: 38 and the second guide sequence employed comprises or consists of a sequence that has 100% sequence identity to at least 17, at least 18, at least 19, or all 20 nucleotides of SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 39, SEQ ID NO: 40, SEQ ID NO: 41, or SEQ ID NO: 43. In some embodiments, the first guide sequence employed comprises or consists of a sequence that has 100% sequence identity to at least 17, at least 18, at least 19, or all 20 nucleotides of SEQ ID NO: 36, SEQ ID NO: 37, or SEQ ID NO: 38 and the second guide sequence employed comprises or consists of a sequence that has 100% sequence identity to at least 17, at least 18, at least 19, or all 20 nucleotides of SEQ ID NO: 8, SEQ ID NO: 16, SEQ ID NO: 19, SEQ ID NO: 27, SEQ ID NO: 35, SEQ ID NO: 39, or SEQ ID NO: 43. In some embodiments, the first guide sequence employed comprises or consists of a sequence that has 100% sequence identity to at least 17, at least 18, at least 19, or all 20 nucleotides of SEQ ID NO: 37 and the second guide sequence employed comprises or consists of a sequence that has 100% sequence identity to at least 17, at least 18, at least 19, or all 20 nucleotides of SEQ ID NO: 16. In some embodiments, the first guide sequence employed comprises or consists of a sequence that has 100% sequence identity to at least 17, at least 18, at least 19, or all 20 nucleotides of SEQ ID NO: 37 and the second guide sequence employed comprises or consists of a sequence that has 100% sequence identity to at least 17, at least 18, at least 19, or all 20 nucleotides of SEQ ID NO: 27. In some embodiments, the first guide sequence employed comprises or consists of a sequence that has 100% sequence identity to at least 17, at least 18, at least 19, or all 20 nucleotides of SEQ ID NO: 37 and the second guide sequence employed comprises or consists of a sequence that has 100% sequence identity to at least 17, at least 18, at least 19, or all 20 nucleotides of SEQ ID NO: 39. In some embodiments, the first guide sequence employed comprises or consists of SEQ ID NO: 37 and the second guide sequence employed comprises or consists of SEQ ID NO: 16. In some embodiments, the first guide sequence employed comprises or consists of SEQ ID NO: 37 and the second guide sequence employed comprises or consists of SEQ ID NO: 27. In some embodiments, the first guide sequence employed comprises or consists of SEQ ID NO: 37 and the second guide sequence employed comprises or consists of SEQ ID NO: 39.

In some embodiments, the first guide sequence employed comprises or consists of a sequence that has 100% sequence identity to at least 17, at least 18, at least 19, or all 20 nucleotides of SEQ ID NO: 5 or SEQ ID NO: 6 and the second guide sequence employed comprises or consists of a sequence that has 100% sequence identity to at least 17, at least 18, at least 19, or all 20 nucleotides of SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 39, SEQ ID NO: 40, SEQ ID NO: 41, or SEQ ID NO: 43. In some embodiments, the first guide sequence employed comprises or consists of a sequence that has 100% sequence identity to at least 17, at least 18, at least 19, or all 20 nucleotides of SEQ ID NO: 5 or SEQ ID NO: 6 and the second guide sequence employed comprises or consists of a sequence that has 100% sequence identity to at least 17, at least 18, at least 19, or all 20 nucleotides of SEQ ID NO: 8, SEQ ID NO: 16, SEQ ID NO: 19, SEQ ID NO: 27, SEQ ID NO: 35, SEQ ID NO: 39, or SEQ ID NO: 43. In some embodiments, the first guide sequence employed comprises or consists of a sequence that has 100% sequence identity to at least 17, at least 18, at least 19, or all 20 nucleotides of SEQ ID NO: 5 and the second guide sequence employed comprises or consists of a sequence that has 100% sequence identity to at least 17, at least 18, at least 19, or all 20 nucleotides of SEQ ID NO: 8. In some embodiments, the first guide sequence employed comprises or consists of SEQ ID NO: 5 and the second guide sequence employed comprises or consists of SEQ ID NO: 8.

In some embodiments, the first guide sequence employed comprises or consists of a sequence that has 100% sequence identity to at least 17, at least 18, at least 19, or all 20 nucleotides of SEQ ID NO: 3 or SEQ ID NO: 4 and the second guide sequence employed comprises or consists of a sequence that has 100% sequence identity to at least 17, at least 18, at least 19, or all 20 nucleotides of SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 39, SEQ ID NO: 40, SEQ ID NO: 41, or SEQ ID NO: 43. In some embodiments, the first guide sequence employed comprises or consists of a sequence that has 100% sequence identity to at least 17, at least 18, at least 19, or all 20 nucleotides of SEQ ID NO: 3 or SEQ ID NO: 4 and the second guide sequence employed comprises or consists of a sequence that has 100% sequence identity to at least 17, at least 18, at least 19, or all 20 nucleotides of SEQ ID NO: 8, SEQ ID NO: 16, SEQ ID NO: 19, SEQ ID NO: 27, SEQ ID NO: 35, SEQ ID NO: 39, or SEQ ID NO: 43.

In some embodiments, the first guide sequence employed comprises or consists of a sequence that has 100% sequence identity to at least 17, at least 18, at least 19, or all 20 nucleotides of SEQ ID NO: 5, SEQ ID NO: 6, SEQ ID NO: 88, SEQ ID NO: 89, SEQ ID NO: 90, SEQ ID NO: 91, SEQ ID NO: 92, SEQ ID NO: 93, SEQ ID NO: 94, or SEQ ID NO: 95 and the second guide sequence employed comprises or consists of a sequence that has 100% sequence identity to at least 17, at least 18, at least 19, or all 20 nucleotides of SEQ ID NO: 7, SEQ ID NO: 8, SEQ ID NO: 9, SEQ ID NO: 96, SEQ ID NO: 97, or SEQ ID NO: 98.

In some embodiments, the first guide sequence employed comprises or consists of a sequence that has 100% sequence identity to at least 17, at least 18, at least 19, or all 20 nucleotides of SEQ ID NO: 36, SEQ ID NO: 37, SEQ ID NO: 38, SEQ ID NO: 111, SEQ ID NO: 112, SEQ ID NO: 113, SEQ ID NO: 114, or SEQ ID NO: 115 and the second guide sequence employed comprises or consists of a sequence that has 100% sequence identity to at least 17, at least 18, at least 19, or all 20 nucleotides of SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 99, SEQ ID NO: 100, SEQ ID NO: 101, SEQ ID NO: 102, or SEQ ID NO: 103.

In some embodiments, the first guide sequence employed comprises or consists of a sequence that has 100% sequence identity to at least 17, at least 18, at least 19, or all 20 nucleotides of SEQ ID NO: 42 and the second guide sequence employed comprises or consists of a sequence that has 100% sequence identity to at least 17, at least 18, at least 19, or all 20 nucleotides of SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 99, SEQ ID NO: 100, SEQ ID NO: 101, SEQ ID NO: 102, or SEQ ID NO: 103.

In some embodiments, the first guide sequence employed comprises or consists of a sequence that has 100% sequence identity to at least 17, at least 18, at least 19, or all 20 nucleotides of SEQ ID NO: 36, SEQ ID NO: 37, SEQ ID NO: 38, SEQ ID NO: 111, SEQ ID NO: 112, SEQ ID NO: 113, SEQ ID NO: 114, or SEQ ID NO: 115 and the second guide sequence employed comprises or consists of a sequence that has 100% sequence identity to at least 17, at least 18, at least 19, or all 20 nucleotides of SEQ ID NO: 22, SEQ ID NO: 23, SEQ ID NO: 24, SEQ ID NO: 25, SEQ ID NO: 26, SEQ ID NO: 27, SEQ ID NO: 106, or SEQ ID NO: 107.

In some embodiments, the first guide sequence employed comprises or consists of a sequence that has 100% sequence identity to at least 17, at least 18, at least 19, or all 20 nucleotides of SEQ ID NO: 42 and the second guide sequence employed comprises or consists of a sequence that has 100% sequence identity to at least 17, at least 18, at least 19, or all 20 nucleotides of SEQ ID NO: 22, SEQ ID NO: 23, SEQ ID NO: 24, SEQ ID NO: 25, SEQ ID NO: 26, SEQ ID NO: 27, SEQ ID NO: 106, or SEQ ID NO: 107.

In some embodiments, the first guide sequence employed comprises or consists of a sequence that has 100% sequence identity to at least 17, at least 18, at least 19, or all 20 nucleotides of SEQ ID NO: 36, SEQ ID NO: 37, SEQ ID NO: 38, SEQ ID NO: 111, SEQ ID NO: 112, SEQ ID NO: 113, SEQ ID NO: 114, or SEQ ID NO: 115 and the second guide sequence employed comprises or consists of a sequence that has 100% sequence identity to at least 17, at least 18, at least 19, or all 20 nucleotides of SEQ ID NO: 39, SEQ ID NO: 40, SEQ ID NO: 41, SEQ ID NO: 116, SEQ ID NO: 117, or SEQ ID NO: 118.

In some embodiments, the first guide sequence employed comprises or consists of a sequence that has 100% sequence identity to at least 17, at least 18, at least 19, or all 20 nucleotides of SEQ ID NO: 42 and the second guide sequence employed comprises or consists of a sequence that has 100% sequence identity to at least 17, at least 18, at least 19, or all 20 nucleotides of SEQ ID NO: 43.

In some embodiments, the first guide sequence employed comprises or consists of a sequence that has 100% sequence identity to at least 17, at least 18, at least 19, or all 20 nucleotides of SEQ ID NO: 42 and the second guide sequence employed comprises or consists of a sequence that has 100% sequence identity to at least 17, at least 18, at least 19, or all 20 nucleotides of SEQ ID NO: 3, or SEQ ID NO: 4.

In some embodiments, the first guide sequence employed comprises or consists of a sequence that has 100% sequence identity to at least 17, at least 18, at least 19, or all 20 nucleotides of SEQ ID NO: 42 and the second guide sequence employed comprises or consists of a sequence that has 100% sequence identity to at least 17, at least 18, at least 19, or all 20 nucleotides of SEQ ID NO: 7, SEQ ID NO: 8, SEQ ID NO: 9, SEQ ID NO: 96, SEQ ID NO: 97, or SEQ ID NO: 98.

In some embodiments, the first guide sequence employed comprises or consists of a sequence that has 100% sequence identity to at least 17, at least 18, at least 19, or all 20 nucleotides of SEQ ID NO: 7, SEQ ID NO: 8, SEQ ID NO: 9, SEQ ID NO: 96, SEQ ID NO: 97, or SEQ ID NO: 98 and the second guide sequence employed comprises or consists of a sequence that has 100% sequence identity to at least 17, at least 18, at least 19, or all 20 nucleotides of SEQ ID NO: 17, SEQ ID NO: 18, SEQ ID NO: 19, SEQ ID NO: 20, SEQ ID NO: 21, SEQ ID NO: 104, or SEQ ID NO: 105.

In some embodiments, the first guide sequence employed comprises or consists of a sequence that has 100% sequence identity to at least 17, at least 18, at least 19, or all 20 nucleotides of SEQ ID NO: 7, SEQ ID NO: 8, SEQ ID NO: 9, SEQ ID NO: 96, SEQ ID NO: 97, or SEQ ID NO: 98 and the second guide sequence employed comprises or consists of a sequence that has 100% sequence identity to at least 17, at least 18, at least 19, or all 20 nucleotides of SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 108, SEQ ID NO: 109, or SEQ ID NO: 110.

In some embodiments, the first guide sequence employed comprises or consists of a sequence that has 100% sequence identity to at least 17, at least 18, at least 19, or all 20 nucleotides of SEQ ID NO: 7, SEQ ID NO: 8, SEQ ID NO: 9, SEQ ID NO: 96, SEQ ID NO: 97, or SEQ ID NO: 98 and the second guide sequence employed comprises or consists of a sequence that has 100% sequence identity to at least 17, at least 18, at least 19, or all 20 nucleotides of SEQ ID NO: 43.

In some embodiments, the first guide sequence employed comprises or consists of a sequence that has 100% sequence identity to at least 17, at least 18, at least 19, or all 20 nucleotides of SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 99, SEQ ID NO: 100, SEQ ID NO: 101, SEQ ID NO: 102, or SEQ ID NO: 103 and the second guide sequence employed comprises or consists of a sequence that has 100% sequence identity to at least 17, at least 18, at least 19, or all 20 nucleotides of SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 108, SEQ ID NO: 109, or SEQ ID NO: 110.

In some embodiments, the first guide sequence employed comprises or consists of a sequence that has 100% sequence identity to at least 17, at least 18, at least 19, or all 20 nucleotides of SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 99, SEQ ID NO: 100, SEQ ID NO: 101, SEQ ID NO: 102, or SEQ ID NO: 103 and the second guide sequence employed comprises or consists of a sequence that has 100% sequence identity to at least 17, at least 18, at least 19, or all 20 nucleotides of SEQ ID NO: 43.

In some embodiments, the first guide sequence employed comprises or consists of a sequence that has 100% sequence identity to at least 17, at least 18, at least 19, or all 20 nucleotides of SEQ ID NO: 22, SEQ ID NO: 23, SEQ ID NO: 24, SEQ ID NO: 25, SEQ ID NO: 26, SEQ ID NO: 27, SEQ ID NO: 106, or SEQ ID NO: 107 and the second guide sequence employed comprises or consists of a sequence that has 100% sequence identity to at least 17, at least 18, at least 19, or all 20 nucleotides of SEQ ID NO: 43.

In some embodiments, the first guide sequence employed comprises or consists of a sequence that has 100% sequence identity to at least 17, at least 18, at least 19, or all 20 nucleotides of SEQ ID NO: 1, SEQ ID NO: 2, or SEQ ID NO: 87 and the second guide sequence employed comprises or consists of a sequence that has 100% sequence identity to at least 17, at least 18, at least 19, or all 20 nucleotides of a sequence selected from the group consisting of SEQ ID NOs: 5-35, 39-41, 43, 88-110, and 116-118.

In some embodiments, the first guide sequence employed comprises or consists of a sequence that has 100% sequence identity to at least 17, at least 18, at least 19, or all 20 nucleotides of SEQ ID NO: 42 and the second guide sequence employed comprises or consists of a sequence that has 100% sequence identity to at least 17, at least 18, at least 19, or all 20 nucleotides of a sequence selected from the group consisting of SEQ ID NOs: 3-35, 39-41, 43, 88-110, and 116-118.

In some embodiments, the first guide sequence employed comprises or consists of a sequence that has 100% sequence identity to at least 17, at least 18, at least 19, or all 20 nucleotides of SEQ ID NO: 36, SEQ ID NO: 37, SEQ ID NO: 38, SEQ ID NO: 111, SEQ ID NO: 112, SEQ ID NO: 113, SEQ ID NO: 114, or SEQ ID NO: 115 and the second guide sequence employed comprises or consists of a sequence that has 100% sequence identity to at least 17, at least 18, at least 19, or all 20 nucleotides of a sequence selected from the group consisting of SEQ ID NOs: 3-35, 39-41, 43, 88-110, and 116-118.

In some embodiments, the first guide sequence employed comprises or consists of a sequence that has 100% sequence identity to at least 17, at least 18, at least 19, or all 20 nucleotides of SEQ ID NO: 5, SEQ ID NO: 6, SEQ ID NO: 88, SEQ ID NO: 89, SEQ ID NO: 90, SEQ ID NO: 91, SEQ ID NO: 92, SEQ ID NO: 93, SEQ ID NO: 94, or SEQ ID NO: 95 and the second guide sequence employed comprises or consists of a sequence that has 100% sequence identity to at least 17, at least 18, at least 19, or all 20 nucleotides of a sequence selected from the group consisting of SEQ ID NOs: 7-35, 39-41, 43, 96-110, and 116-118.

In some embodiments, the second guide sequence employed comprises or consists of a sequence that has 100% sequence identity to at least 17, at least 18, at least 19, or all 20 nucleotides of SEQ ID NO: 43 and the first guide sequence employed comprises or consists of a sequence that has 100% sequence identity to at least 17, at least 18, at least 19, or all 20 nucleotides of a sequence selected from the group consisting of SEQ ID NOs: 1-38, 42, 87-115.

In some embodiments, the second guide sequence employed comprises or consists of a sequence that has 100% sequence identity to at least 17, at least 18, at least 19, or all 20 nucleotides of SEQ ID NO: 39, SEQ ID NO: 40, SEQ ID NO: 41, SEQ ID NO: 116, SEQ ID NO: 117, or SEQ ID NO: 118 and the first guide sequence employed comprises or consists of a sequence that has 100% sequence identity to at least 17, at least 18, at least 19, or all 20 nucleotides of a sequence selected from the group consisting of SEQ ID NOs: 1-38, 42, 87-115.

In some embodiments, the second guide sequence employed comprises or consists of a sequence that has 100% sequence identity to at least 17, at least 18, at least 19, or all 20 nucleotides of SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 108, SEQ ID NO: 109, or SEQ ID NO: 110 and the first guide sequence employed comprises or consists of a sequence that has 100% sequence identity to at least 17, at least 18, at least 19, or all 20 nucleotides of a sequence selected from the group consisting of SEQ ID NOs: 1-21, 36-38, 42, 87-105, and 111-115.

In some embodiments, the second guide sequence employed comprises or consists of a sequence that has 100% sequence identity to at least 17, at least 18, at least 19, or all 20 nucleotides of SEQ ID NO: 22, SEQ ID NO: 23, SEQ ID NO:, 24, SEQ ID NO: 25, SEQ ID NO: 26, SEQ ID NO: 27, SEQ ID NO: 106, or SEQ ID NO: 107 and the first guide sequence employed comprises or consists of a sequence that has 100% sequence identity to at least 17, at least 18, at least 19, or all 20 nucleotides of a sequence selected from the group consisting of SEQ ID NOs: 1-21, 36-38, 42, 87-105, and 111-115.

In some embodiments, the second guide sequence employed comprises or consists of a sequence that has 100% sequence identity to at least 17, at least 18, at least 19, or all 20 nucleotides of SEQ ID NO: 17, SEQ ID NO: 18, SEQ ID NO: 19, SEQ ID NO: 20, SEQ ID NO: 21, SEQ ID NO: 104, or SEQ ID NO: 105 and the first guide sequence employed comprises or consists of a sequence that has 100% sequence identity to at least 17, at least 18, at least 19, or all 20 nucleotides of a sequence selected from the group consisting of SEQ ID NOs: 1-9, 36-38, 42, 87-98, and 111-115.

In some embodiments, the second guide sequence employed comprises or consists of a sequence that has 100% sequence identity to at least 17, at least 18, at least 19, or all 20 nucleotides of SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 99, SEQ ID NO: 100, SEQ ID NO: 101, SEQ ID NO: 102, or SEQ ID NO: 103 and the first guide sequence employed comprises or consists of a sequence that has 100% sequence identity to at least 17, at least 18, at least 19, or all 20 nucleotides of a sequence selected from the group consisting of SEQ ID NOs: 1-9, 36-38, 42, 87-98, and 111-115.

In some embodiments, the second guide sequence employed comprises or consists of a sequence that has 100% sequence identity to at least 17, at least 18, at least 19, or all 20 nucleotides of SEQ ID NO: 7, SEQ ID NO: 8, SEQ ID NO: 9, SEQ ID NO: 96, SEQ ID NO: 97, or SEQ ID NO: 98 and the first guide sequence employed comprises or consists of a sequence that has 100% sequence identity to at least 17, at least 18, at least 19, or all 20 nucleotides of a sequence selected from the group consisting of SEQ ID NOs: 1-6, 36-38, 42, 87-95, and 111-115.

In some embodiments, the Cas protein or a nucleotide sequence encoding the Cas protein and/or one or more gRNAs (or encoding nucleotide molecule(s) thereof) are delivered to the cell or the subject in the form of a nanoparticle, e.g., as a complex with a nanocarrier. Exemplary nanocarriers include organic and inorganic nanocarriers known in the art. Particularly preferred nanocarriers include polyrotaxane (PRX) nanocarriers.

In some embodiments, the one or more gRNAs are delivered to a subject in the form of a complex with a Cas protein (as described herein), e.g., Cas9. In some embodiments, the one or more gRNAs are delivered to a cell, such as a stem cell (e.g., an iPSC), or a subject in the form of a viral vector, e.g., an AAV vector.

In some embodiments, the present invention is directed to treating Duchenne muscular dystrophy or Becker muscular dystrophy in a subject which comprises modifying the dystrophin gene in the subject as described above. In some embodiments, the subject is human. In some embodiments, the subject is a test animal, e.g., a mouse, that has been genetically modified to carry a DMD gene to be modified.

### Examples

### Guide RNAs

The guide sequences as set forth in Table 1 were designed to target the introns and exons of the DMD gene as indicated in FIG. 1 using the Zhang lab CRISPR design tool (“crispr.mit“followed by”.edu”) or CRISPOR and cloned into the spCas9 plasmids pX330 or pX459 from Feng Zhang (Addgene #42230, #48139 respectively) adapted from Ran et al. (Nature Protocols, 2013. 8:2281-2308). In brief, oligos complementary to each other containing the gRNA sequence and BbsI restriction enzyme site were obtained from Integrated DNA Technologies and annealed. The annealed oligos and plasmid were simultaneously digested with BbsI (New England Biotechnologies, Ipswich, Mass.) and ligated with T4 DNA ligase (Thermo Fisher Scientific, Waltham, Mass.). Dual gRNAs were cloned into pX333 from Andrea Ventura (Addgene plasmid 6403) with BbsI and BsaI.

### Mice

All animal work was conducted under protocols approved by the UCLA Animal Research Committee in the Office of Animal Research Oversight. Mice used for engraftment experiments are mdx NSG mice, obtained by crossing mdx scid (Jackson Laboratory, Bar Harbor, Me.) with NOD scid IL2Rgamma (Jackson Laboratory, Bar Harbor, Me.) mice. Genotyping is performed through TransnetYX. Mice that were used for AAV delivery in vivo, hDMD del45 mdx mice, were maintained and genotyped as described (Young et al. J Neuromuscl Dis. 2017; 4(2): 139-145).

### Cell Culture

Human embryonic kidney (HEK) 293FT and 293AAV cells (Thermo Fisher Scientific, Waltham, Mass.) were grown in standard conditions with growth medium consisting of DMEM (high glucose) with 10% fetal bovine serum (FBS, Thermo Fisher Scientific, Waltham, Mass.), 0.1 mM non-essential amino acids (NEAA, Thermo Fisher Scientific, Waltham, Mass.), 6 mM L-glutamine (Thermo Fisher Scientific, Waltham, Mass.).

Human skeletal muscle myoblasts (HSMM, Lonza, Basel Switzerland) are maintained according to the manufacturer's instructions with SkGM-2 medium (Lonza, Basel Switzerland). For terminal differentiation, they are cultured on Matrigel (Corning, Corning N.Y.) until at least 80% confluent and then switched to N2 differentiation medium (DMEM/F12 with 1% N2 supplement (Thermo Fisher Scientific, Waltham, Mass.) and 1% insulin-transferrin-selenium (ITS, Thermo Fisher Scientific, Waltham, Mass.)) for 7 days.

Human induced pluripotent stem cells (hiPSCs) are reprogrammed from skin fibroblasts with the STEMCCA cassette as previously described (Karumbayaram et al., Stem Cells Transl Med., 2012, 1:36-43). They are grown on hESC qualified Matrigel (Corning, Corning N.Y.), fed daily with mTeSR1 medium (Stem Cell Technologies, Vancouver Canada) and passaged with 0.5 mM EDTA every 5-7 days. Karyotype and FISH analyses are performed by Cell Line Genetics (Madison, Wis.).

Primary hDMD or hDMD del45 mdx myoblasts are obtained from 11-13 day old pups by dissociation of muscle tissue using a 1:1 mixture of 1.5 mg/mL dispase (neutral protease, Worthington Biochemical, Lakewood N.J.) and 1600 U/mL collagenase II (Worthington Biochemical, Lakewood N.J.) in PBS at 200 μl per 100 mg tissue. Muscles are minced, then incubated at 37° C. with slow agitation for 30 minutes. Fibroblasts were removed by repeatedly pre-plating. Myoblasts are cultured on entactin-collagen IV-laminin cell attachment matrix (ECL, EMD Millipore, Burlington Mass.) and are maintained in F-10 HAM (Sigma-Aldrich, St Louis Mo.) with 20% FBS, 5 ng/mL basic fibroblast growth factor (bFGF, Promega, Madison Wis.) and 1% penicillin/streptomycin (P/S, Thermo Fisher Scientific, Waltham, Mass.). Myoblasts are differentiated to form myotubes (at >80% confluence) in DMEM (Thermo Fisher Scientific, Waltham, Mass.) with 2% horse serum (Thermo Fisher Scientific, Waltham, Mass.), 1% ITS and 1% P/S on Matrigel® basement membrane matrix (Corning, Corning N.Y.).

### Teratoma Injections

To prepare hiPSCs for injection, 1-2 confluent wells are collected using 1 mg/ml of collagenase type IV or 0.5 mM EDTA. Colonies are dissociated using a 5 ml pipette and centrifuged at 1000 rpm. Cell pellets are resuspended in 40 μl of Hank's Balanced Salt Solution (HBSS) and injected into the testes of 6-8 week-old SCID BEIGE mice (Charles River, Wilmington Mass.) as described previously (Alva et al., Stem Cells, 2011, 29:1952-1962). After 4-8 weeks, teratomas are isolated and fixed in 4% paraformaldehyde (PFA) for 24 hours, then 70% ethanol. Fixed teratomas are embedded and processed by the Tissue Procurement Core Laboratory, Department of Pathology and Laboratory Medicine, David Geffen School of Medicine at UCLA. If teratomas are large, four to six quadrants are isolated and fixed in PFA as described above. Tumors are stained with hematoxylin and eosin and imaged with an Olympus BX51 microscope.

Transfection and Nucleofection of gRNAs

3×10⁵ HEK293FT cells were seeded per 12 well and transfected in duplicate or triplicate the following day with 1 μg DNA using 3 μl TransIT293 (Mirus Bio, Madison Wis.) or ViaFect (Promega, Madison Wis.) according to the manufacturer's instructions.

Amaxa 4D (Lonza, Basel Switzerland) nucleofection of hiPSCs is performed according to the manufacturer's instructions. In brief, hiPSCs are pre-treated with 10 μM ROCK inhibitor Y-27632 (ROCKi, Tocris Bioscience, Bristol UK) for 1 hour and trypsinized into a single cell suspension with TryplE (Thermo Fisher Scientific, Waltham, Mass.). 8×10⁵ hiPSCs are nucleofected per 100 μl cuvette using solution P3, 2 μg or 3.5 μg total DNA, and program CA-137 (Lonza). pMAX GFP (Lonza) is used as a transfection control. After nucleofection, cells are immediately plated in mTeSR1 with ROCKi. For selection, 0.35 μg/ml of puromycin in mTeSR1 is added to the cells for 24 hours the day after nucleofection.

Generation of Clonal hiPSC Lines

For generation of clonal lines, the day following nucleofection of gRNAs (in pX459) cells are selected with 0.35 μg/ml of puromycin in mTeSR1 for one day. The cells are expanded for 7-9 days in mTeSR1 and then either single cell sorting in ROCKi using a FACSAria sorter (BD, Franklin Lakes, N.J.) into individual 96 wells or plating at low densities of 3×10⁵-5×10⁵ cells per 10 cm dish in mTeSR1 plus ROCKi occurs. After 2 weeks, individual colonies are scrapped into a corresponding 48 well and a subset of the colony is manually dissected and screened using the deletion genotyping PCR below.

### Deletion Genotyping PCR

For determining if a desired deletion in the DMD gene occurred, either individual PCR reactions or a multiplex PCR containing both sets of primers is performed with AccuPrime Taq High Fidelity (Thermo Fisher Scientific, Waltham, Mass.). One primer pair flanks the deleted region (del) and one pair is within the deleted region (undel). PCR products are run on a 1.2% agarose gel and visualized with ethidium bromide staining.

Analysis of the rejoining sequence after the desired deletion is performed by blunt cloning the deleted PCR products into the Zero Blunt TOPO backbone (Thermo Fisher Scientific, Waltham, Mass.), according to the manufacturer's instructions, and sequencing of the insert by Laragen Inc (Culver City, Calif.).

Differentiation of hiPSC-Derived Skeletal Muscle

hiPSCs are differentiated into skeletal muscle cells by overexpression of MyoD, adapted from Abujarour et al. (Stem Cells Trans Med., 2014, 3:149-160). Cells are trypsinized with TryplE and plated as single cells on Matrigel in SMC4 (basal medium: DMEM/F-12 with 20% knock-out serum replacement (KOSR, Thermo Fisher Scientific, Waltham, Mass.), 1% NEAA, 1% Glutamax (Thermo Fisher Scientific, Waltham, Mass.), 100 μM beta-mercaptoethanol, 10 ng/mL basic fibroblast growth factor (bFGF, Thermo Fisher Scientific, Waltham, Mass.); SMC4: basal media with daily addition of 5 μM ROCKi, 0.4 μM PD0325901 (Sigma-Aldrich, St Louis Mo.), 2 μM SB431542 (Tocris Bioscience, Bristol UK), 1 μM CHIR99021 (Tocris Bioscience, Bristol UK)) at 3.5×10⁵ cells/6 well. When they reach approximately 60-80% confluent they are infected with 0.06 μg/mL of a tamoxifen inducible MyoD-ERT lentivirus (adapted from Kimura et al., Hum Mol Genet, 2008, 17:2507-2517) with 4 μg/mL protamine sulfate per well and spun inoculated at 1250 rpm for 90 minutes at 32° C. After a day of recovery they are selected with 2 μg/ml puromycin in SMC4 for 2 days. The cells are then split and plated on Matrigel in basal medium without bFGF plus 10 μM ROCKi at approximately 1×10⁵ cells/cm² and induced in DMEM with 15% FBS and 5 μM tamoxifen for 4 days. Following induction, the cells are differentiated in low glucose DMEM with 5% horse serum and 1 μM tamoxifen for 5-7 days. Medium is changed daily.

An alternative protocol for MyoD overexpression may be used for engraftment. Cells are single cell plated at 2.5×10⁴ cells/cm² on Matrigel in mTeSR1 with ROCKi. Beginning the following day, they are treated with 3 μM CHIR99021 in DMEM/F12 with 1% ITS for 2 days. The cells are split to approximately 6×10⁴ cells/cm² in DMEM with 10% FBS and 1% NEAA and infected with the MyoD-ERT lentivirus as above. After a day of recovery, the cells are selected with 1 μg/ml puromycin for 4 days followed by induction in IMDM containing 15% FBS, 10% horse serum (HS), 1% chick embryo extract, 50 μg/ml ascorbic acid, 4.5 mM monothioglycerol, 5 ng/ml bFGF with 5 μM tamoxifen for 2 days and used for engraftment as described below.

A directed differentiation protocol for hiPSCs adapted from Shelton et al. (Stem Cell Reports, 2014, 3:516-529) was also used to obtain SMPCs as described in Hicks, et al. (2017) Nat Cell Biol 20:46-5. Cells were single cell plated in mTeSR1 with 10 μM ROCKi at 3.75×10⁵ cells/6 well. The following day, 10 μM of CHIR99021 was added in Essential 6 medium (E6, Stem Cell Technologies, Vancouver Canada) for 2 days and the cells were allowed to differentiate until day 12 in E6. StemPro (Thermo Fisher Scientific, Waltham, Mass.) containing 20 ng/ml bFGF was added between days 12 to 20. E6 was then added until day 35 when the cells were switched to N2 medium in order to terminally differentiate. At day 50, cells were fluorescently activated cell sorted to remove neural crest cells with HNK1⁻ (1:300, Sigma-Aldrich, St Louis Mo.) and enrich for SMPCs with ERBB3 and NGFR. The SMPCs were cultured in expansion media (20% FBS, 5% HS, 1% chick embryo extract, 0.5% penicillin/streptomycin) until confluent when they were changed to low glucose DMEM with 2% horse serum and 1% ITS for 5-7 days to induce terminal differentiation.

Differentiation of hiPSC-Derived Cardiomyocytes

Confluent hiPSCs are enzymatically dissociated to form aggregates and differentiated into the cardiomyocyte lineage using methods in the art. The medium is changed every 2 days up to day 15, and every 5 days up to day 30. At day 30, cardiomyocytes are harvested for analysis or subjected to the hypoosmotic stress assay.

### Assessment of Editing Efficiency

For testing the activity of different gRNAs, genomic DNA (gDNA) was extracted on day 3 or 4 after transfection/nucleofection using the Quick gDNA mini prep kit (Zymo Research, Irvine Calif.) or Quick Extract DNA Extraction Solution 1.0 (Epicenter, Lucigen, Middleton Wis.) according to the manufacturer's instructions. PCR for use in TIDE or Surveyor assays was performed with AccuPrime Taq High Fidelity with primers flanking the target region.

The PCR products were sequenced by Laragen Inc and TIDE performed using software at https://tide.deskgen.com/ as described (Brinkman Nucleic Acids Research 2014). The Surveyor assay (Integrated DNA Technologies, Coralville, Iowa) is performed according to the manufacturer's instructions. In brief, approximately 300 ng of PCR product in 1× AccuPrime buffer up to 20 μl is denatured and reannealed by heating at 95° C. for 10 minutes and slowly step-wise cooling to 4° C. Then 2 μl MgCl₂, 1 μl Surveyor enhancer, and 1.2 μl Surveyor enzyme are added and incubated at 42° C. for 1 hour. The G/C plasmids provided in the Surveyor kit are used as a positive control for every gel. The products are run on a 6% or 4-20% TBE polyacrylamide gel (Bio-Rad) and visualized with ethidium bromide staining. The percent of cutting is determined using ImageJ (Rasband, ImageJ, US Natl Institutes Heal, 1997).

### Hypoosmotic Stress CK Release Assay

Terminally differentiated skeletal muscle cells and cardiomyocytes plated in duplicate are stressed by incubation in hypoosmolar solutions ranging from 66-240 mosmol. Hypoosmolar salt solutions ranging from 66-240 mosmol are made by adding varying amounts of sucrose (about 25-175 mM) to a basic salt solution consisting of 5 mM HEPES, 5 mM KCl, 1 mM MgCl₂, 5 mM NaCl, 1.2 mM CaCl₂), 1 mM glucose. Osmolarities are measured with a Wescor Vapro 5520 osmometer.

Differentiated MyoD OE skeletal myotubes and cardiomyocytes are plated in a 384 or 96 well plate in duplicate per condition tested. 100 μl (or 30 μl for 384 well plates) of the hypoosmolar solution is added to each well and the cells are incubated at 37° C. for 20 minutes. The solution (supernatant) is then removed and stored at −80° C. until CK analysis. The cells are trypsinized and lysed in 100 μl dI water by repeated freeze/thawing three times. The lysate is stored at −80° C. until CK analysis. CK is measured in triplicate with 2 μl or 8 μl of undiluted sample using the Creatine Kinase-SL kit (Sekisui Diagnostics, San Diego Calif.) according to the manufacturer's instructions. Any negative readings are forced to be 0 and outliers are discounted from the analysis. The percent of CK release into the supernatant is determined and the standard error is propagated throughout all calculations.

RNA Extraction, cDNA, and PCR

RNA is extracted from differentiated cardiomyocytes using the RNeasy Micro Kit (Qiagen, Hilden Germany) according to the manufacturer's instructions. cDNA synthesis is performed on 50-250 ng of RNA using the iScript Reverse Transfection Supermix for RT-qPCR (Bio-Rad, Hercules Calif.). Two PCR reactions are done on all samples, one with primers internal to the deletion and one with primers flanking the deletion for 40 cycles using AccuPrime Taq. PCR products are cloned using the TOPO-TA cloning kit (Thermo Fisher Scientific, Waltham, Mass.) according to the manufacturer's instructions and sequenced at Laragen Inc.

miRNA Extraction, cDNA, and ddPCR

miRNA is isolated from fused myotubes obtained by MyoD OE using a microRNA purification kit (Norgen Biotek Corp, Thorold Canada) according to the manufacturer's instructions. cDNA synthesis is performed on 5 μl of miRNA with TaqMan microRNA reverse transcription kit (Applied Biosystems, Foster City Calif.) using a TaqMan MicroRNA Assay (Applied Biosystems, Foster City Calif.) for hsa-miR-31 (assay ID 002279) and U6 snRNA (assay ID 001973) with specific RT primers. PCR reactions are prepared in a premix of 22 μl with 1.46 μl of cDNA (either diluted 1:30 for U6 or undiluted for miR31) in ddPCR supermix for probes (without UTP) (Bio-Rad, Hercules Calif.) and 1.1 μl 20× TaqMan assay probes for each sample in duplicate. 20 μl of the PCR reaction premix is used to generate droplets according to the manufacturer's protocol. Briefly, PCR premix is added to a droplet generator cartridge with 70 μl of oil and droplets are generated with the QX200 droplet generator (Bio-Rad, Hercules Calif.). 40 μl of this reaction mix is transferred to a PCR plate and run on at T100 thermal cycler (Bio-Rad, Hercules Calif.) at 95° C. for 10 minutes, 40 cycles of 95° C. for 15 seconds, 60° C. for 60 seconds, followed by 98° C. for 10 minutes. A no template control is included for each PCR reaction. FAM fluorescence is evaluated using the QX200 droplet reader and QuantaSoft software (Bio-Rad, Hercules Calif.). The percent of positive droplets for miR31 is normalized to the percent of positive droplets for U6. Standard deviation error is propagated through all calculations. All lines are normalized to wild type.

qPCR Assessment of Exon 46 Deletion

TaqMan qPCR was performed in multiplex according to the manufacturer's instructions using primers flanking DMD exons 46 and 17 as well as a FAM fluorescence probe targeted to DMD exon 46 and a HEX probe targeted to exon 17. Each sample was run in triplicate on 30 ng of DNA and evaluated with the CFX Connect qPCR machine (BioRad, Hercules Calif.).

Engraftment into Immunodeficient Mice

NOD scid IL2Rgamma (NSG) immunodeficient mice (Jackson Laboratory Bar Harbor, Me.) are crossed to mdx scid mice (Jackson Laboratory Bar Harbor, Me.) to generate NSG-mdx mice, see above. 24 hours prior to engraftment, the right tibialis anterior (TA) of 5-7 week-old NSG-mdx mice is pretreated with 50 μl of 10 μM cardiotoxin (Sigma-Aldrich, St Louis Mo.). For MyoD OE cells, 100 μL of 5 mg/ml tamoxifen (Sigma-Aldrich, St Louis Mo.) is also IP injected for 5 days with tamoxifen pretreatment starting the day before engraftment (Muir et al., Mol Ther Methods Clin Dev, 2014, 1:14025). 1×10⁶ cells obtained from MyoD OE after induction are pelleted and resuspended in 5 μL HBSS and injected intramuscularly into the TA. Tissue is harvested after 30 days and analyzed as described below. Engraftment is considered successful when human cells that are both lamin A/C and spectrin positive are identified. Successful engraftment is seen in the following: Cells could also be engrafted systemically.

### Immunostaining

hiPSCs are fixed in 4% PFA for 20 minutes, permeabilized with 0.3% Triton X for 10 minutes and blocked in 10% goat serum for 1 hour. Primary antibodies to SOX2 (1:200, Cell Signaling, Danvers Mass.) and NANOG (1:800, Cell Signaling, Danvers Mass.) are added in 1% goat serum and 0.1% Triton X overnight at 4° C. followed by secondary antibodies for 2 hours the following day.

Differentiated skeletal myotubes obtained from MyoD overexpression are fixed in 80% acetone for 7 minutes at −20° C., blocked with 10% goat serum for 1 hour and stained with dystrophin (1:300, Abcam, Cambridge UK) and myosin heavy chain (1.9 μg/ml, MF20, DSHB, Iowa City Iowa) as above. Differentiated cardiomyocytes and skeletal myotubes obtained from the 50 day directed differentiation protocol were fixed in 4% PFA for 20 minutes, permeabilized with 0.3% Triton X for 10 minutes, blocked in 10% goat serum for 1 hour and stained with dystrophin (1:5, MANDYS106, MDA Monoclonal Antibody Resource, Gobowen UK (Man and Morris, Am J Hum Genet, 1993, 52:1057-1066)) or beta-dystroglycan (1:100, Leica Biosystems, Wetzlar Germany) and myosin heavy chain (1.9 μg/ml, MF20, DSHB, Iowa City Iowa). Images were obtained with the Axio Observer Z1 microscope (Zeiss, Oberkochen Germany).

Harvested muscles were flash frozen in isopentane. 10 μm cryosections were obtained at intervals throughout the entire muscle and stored at −20° C. For staining, they were blocked in 0.25% gelatin, 0.1% Tween, 3% bovine serum albumin or PBS with 10% goat serum and 5% horse serum for 1 hour. The M.O.M. blocking kit (Vector Laboratories, Burlingame Calif.) is applied according to the manufacturer's instructions. Primary antibodies including dystrophin (1:250, Abcam, Cambridge UK), laminin (1:20, R&D Systems, Minneapolis Minn.), dystrophin 48/50B (1:20, DSHB Iowa City Iowa) were applied overnight at 4° C. Primary antibodies could also consist of human lamin A/C (1:125, Vector Laboratories), human spectrin (1:75, Leica Biosystems, Burlingame Calif.), human dystrophin (1:5, MANDYS106), laminin (1:200, Sigma-Aldrich, St Louis Mo.), and beta-dystroglycan (1:50, Leica Biosystems, Wetzlar Germany) which are applied overnight at 4° C. The following day secondary antibodies were incubated for 1 hour and the slides were mounted with VECTASHIELD containing DAPI (Vector Laboratories, Burlingame Calif.) and imaged on the Axio Observer Z1 microscope.

### Western Blot Analysis

Terminally differentiated skeletal muscle cells and cardiomyocytes are trypsinized, pelleted, and flash frozen in liquid nitrogen. Cell pellets are stored in liquid nitrogen until lysis. For Western blotting, samples are prepared as described in Woo et al. (Exp Mol Med, 2010, 42:614-627) with slight modifications. In brief, cells are solubilized in 500 μl of lysis buffer (10 mM Tris-HCl (pH 7.4), 1% Triton X-100, 10% glycerol, 150 mM NaCl, 5 mM EDTA, and HALT protease and phosphatase inhibitor cocktail (Thermo Fisher Scientific, Waltham, Mass.)) per a 10 cm culture dish, followed by incubation at 4° C. for 30 minutes with gentle rotation. Then lysates are mixed with 100 μl of 6×RSB and passed several times through a syringe needle to reduce viscosity. Afterwards samples are boiled for 3 minutes, cooled on ice, passed through a syringe needle again and centrifuged for 5 minutes at 13,000 g. Clarified lysates are transferred to new tubes, aliquoted and stored at −80° C. until use. To evaluate dystrophin and MyHC content, cell lysates are subjected to 6% polyacrylamide gel electrophoresis (PAGE) for 3 hours at constant current (10 mA per gel); followed by blotting to nitrocellulose membrane at constant voltage (100 V) for 2.5 hours on ice. 0.1% sodium dodecyl sulfate (SDS) and 10 mM dithiothreitol (DTT) is added to the transfer buffer to facilitate blotting of high molecular proteins. Immunoblot assay is carried out with mouse anti-MyHC (1:1,000; MF20, DSHB, Iowa City Iowa), and mouse anti-dystrophin (1:500; Mandys8, Sigma-Aldrich, St Louis Mo.) antibodies. Secondary antibodies used are anti-mouse peroxidase conjugates from Sigma-Aldrich (St Louis Mo., 1:10,000). Blots are developed using ChemiGlow West chemiluminescent detection kit (ProteinSimple, San Jose Calif.). Signals are registered by the FluorChem FC2 digital imaging system (Alpha Innotech, San Leandro, Calif.).

For β-dystroglycan, a 7.5% PAGE gel is run for 1.5 hours at 100 V. Transfer is performed in Tris/Glycine with 20% MetOH for 1.25 hours at 100 V. Immunoblotting is performed with mouse anti-β-dystroglycan (1:200, MANDAG2(7D11), DSHB, Iowa City Iowa) and MyHC antibody as above.

For muscle tissue Western blots, either 100 μg of frozen tissue was solubilized in 50 mM tris-HCl (pH 7.4), 7M urea, 2M thiourea, 4% CHAPS, 2% SDS, 50 mM 0-mercaptoethanol or muscle tissue was homogenized for 1 minute in 1 mL of ice-cold Mito buffer (0.2 mM EDTA, 0.25 mM sucrose, 10 mM tris-HCl (pH 7.4)) with protease/phosphatase inhibitor cocktail (Pierce) and deoxyribonuclease/ribonuclease and subjected to low-speed (1500 g) centrifugation for 10 minutes at 4° C. The supernatant was centrifuged at 100,000 g for 30 minutes for isolation of membrane fraction. Isolated membranes and pellet after low-speed centrifugation were combined and resuspended in 300 μl extraction buffer (50 mM tris-HCl (pH 7.4), 7M urea, 2M thiourea, 4% CHAPS, 2% SDS, 50 mM β-mercaptoethanol) followed by centrifugation for 5 minutes at 13,000 g. Lysates were incubated at 4° C. for 60 minutes with gentle rotation, and centrifuged for 5 minutes at 13,000 g. Clarified lysates were transferred into new tubes, aliquoted and stored at −80° C. until use. Protein concentration was determined using 2-D Quant Kit (GE Healthcare Life Sciences). Muscle lysate were subjected to electrophoresis in a 6% polyacrylamide gel (PAAG), transferred to a nitrocellulose membrane and blotted with anti-human dystrophin antibody MANDYS106 (1:100 in PBSAT, Millipore Sigma, Burlington Mass.) and stained with Ponceau S (Sigma-Aldrich, St Louis Mo.).

### Off Target Analysis

The top unique off target sites for each gRNA used may be determined with COSMID (Cradick et al., Mol Ther Nucleic Acids, 2014, 3:e214) using the following criteria: NRG PAM, 3 mismatches with no indels, and 2 mismatches with 1-base deletions or insertions. Access Array primers corresponding to the potential off target locations are designed, manufactured, and validated by Fluidigm. gDNA extracted from the parental and deleted clonal lines is run on an Access Array (Fluidigm, South San Francisco Calif.) and sequenced with MiSeq in the UCLA GenoSeq Core. Reads are trimmed with Trimmomatic and aligned to the genome using BWA. A base quality score recalibration and indel realignment is performed using GATK and SNP calling is done using two separate programs, GATK and LoFreq on the 20 bp gRNA homologous region. A true induced mutation is considered possible if the fraction of reads with a given variant is substantially higher than error rate of base calling.

### PRX Delivery In Vitro

Myoblasts are seeded at 1.2×10⁵ cells/cm² for growth conditions or 1.7×10⁵ cells/cm² for differentiation where the media is changed to differentiation media the following day. PRX complexed with a CRISPR plasmid is added to the cells at various PRX to plasmid ratios determined empirically.

### PRX Delivery In Vivo

Intramuscular injections of PRX are done into the tibialis anterior (TA) muscle using 2 μg plasmid complexed with PRX into hDMD del45 mdx mice. HBSS is injected into the control mouse. Muscles are harvested and flash frozen in isopentane after 4 weeks and dystrophin is assessed as above. Systemic injections of 50 or 100 μg CRISPR plasmid complexed with PRX is injected into the tail vein of hDMD del45 mdx mice. Mice are dosed 1-4 times over 3 weeks. Muscles are harvested and flash frozen in isopentane and dystrophin is assessed as above.

### AAV Production

gRNAs were cloned into the AAV backbone using the NEBuilder HiFi kit (New England Biotechnologies, Ipswich, Mass.). The target plasmid along with pHelper and pRC9 were transfected into HEK 293AAV cells with TransIt VirusGen (Mirus Bio, Madison Wis.). Medium was changed to serum free after 16 hours and media and pellet were collected 3-5 days post-transfection. AAV was purified using iodixanol centrifugation or will be purified using POROS Capture Select Resin (Thermo Fisher Scientific, Waltham, Mass.) or purchased from a company such as Virovek Inc (Hayward Calif.).

### AAV Delivery In Vivo

Intramuscular delivery of 5×10¹⁰ v.g. of each vector was injected into the TA of hDMD del45 mdx mice. Muscles were harvested 4-5 weeks post-injection. For systemic delivery, 5×10¹¹ v.g. of each vector was injected intraperitoneally (i.p.) into p5 hDMD del45 mdx mice. Doses from 5×10¹¹-1×10¹³ v.g. of each vector can be used through retro-orbital (r.o.) or i.p. injection. Single or multiple injections are given. Muscles were harvested 5-7 weeks later and dystrophin assessed as above.

### Statistical Analysis

Statistical analyses were performed using a two-tailed t-test on two groups of data. First an F-test was used to determine if the variances were equal or unequal, than the corresponding t-test was used. Significance was determined by a p-value less than 0.05.

All scientific and technical terms used in this application have meanings commonly used in the art unless otherwise specified.

As used herein, “Cas proteins” refer to CRISPR associated proteins employed in CRISPR-Cas systems. See, e.g., Makarova, et al. (2015) Nature Reviews Microbiology, 13(11):722-736. Cas proteins include high-fidelity Cas proteins in the art. See, e.g., Kleinstiver, et al., (2018) Nature 529(7587):490-495; Slaymaker, et al., (2016) Science. 351(6268):84-88, and the like. Cas proteins include CRISPR/Cas endonucleases as described in PCT/US2017/017255 and other CRISPR associated proteins in the art. In some embodiments, the Cas proteins are composed of split proteins that join together to form a functional CRISPR/Cas endonuclease. See, e.g., Wright, et al. (2015) PNAS USA 112(10):2984-2989, US 20170298330, and US 20160177278. In some embodiments, the Cas proteins are Cas9 proteins as described in PCT/US2017/017255. In some embodiments, the Cas proteins are Cpfl proteins as described in PCT/US2017/017255. In some embodiments, the Cas proteins are C2c1 proteins as described in PCT/US2017/017255. In some embodiments, the Cas proteins are C2c2 proteins as described in PCT/US2017/017255. In some embodiments, the Cas proteins are C2c3 proteins as described in PCT/US2017/017255.

As used herein, a given percentage of “sequence identity” refers to the percentage of nucleotides or amino acid residues that are the same between sequences, when compared and optimally aligned for maximum correspondence over a given comparison window, as measured by visual inspection or by a sequence comparison algorithm in the art, such as the BLAST algorithm, which is described in Altschul et al., (1990) J Mol Biol 215:403-410. Software for performing BLAST (e.g., BLASTP and BLASTN) analyses is publicly available through the National Center for Biotechnology Information (ncbi.nlm.nih.gov). The comparison window can exist over a given portion, e.g., a functional domain, or an arbitrarily selection a given number of contiguous nucleotides or amino acid residues of one or both sequences. Alternatively, the comparison window can exist over the full length of the sequences being compared. For purposes herein, where a given comparison window (e.g., over 80% of the given sequence) is not provided, the recited sequence identity is over 100% of the given sequence. Additionally, for the percentages of sequence identity of the proteins provided herein, the percentages are determined using BLASTP 2.8.0+, scoring matrix BLOSUM62, and the default parameters available at blast.ncbi.nlm.nih.gov/Blast.cgi. See also Altschul, et al., (1997) Nucleic Acids Res 25:3389-3402; and Altschul, et al., (2005) FEBS J 272:5101-5109.

Optimal alignment of sequences for comparison can be conducted, e.g., by the local homology algorithm of Smith & Waterman, Adv Appl Math 2:482 (1981), by the homology alignment algorithm of Needleman & Wunsch, J Mol Biol 48:443 (1970), by the search for similarity method of Pearson & Lipman, PNAS USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection.

As used herein, the terms “subject”, “patient”, and “individual” are used interchangeably to refer to humans and non-human animals. The term “non-human animal” includes all vertebrates, e.g., mammals and non-mammals, such as non-human primates, horses, sheep, dogs, cows, pigs, chickens, and other veterinary subjects and test animals. In some embodiments, the subject is a mammal. In some embodiments, the subject is a human.

As used herein, the term “diagnosing” refers to the physical and active step of informing, i.e., communicating verbally or by writing (on, e.g., paper or electronic media), another party, e.g., a patient, of the diagnosis. Similarly, “providing a prognosis” refers to the physical and active step of informing, i.e., communicating verbally or by writing (on, e.g., paper or electronic media), another party, e.g., a patient, of the prognosis.

The use of the singular can include the plural unless specifically stated otherwise. As used in the specification and the appended claims, the singular forms “a”, “an”, and “the” can include plural referents unless the context clearly dictates otherwise.

As used herein, “and/or” means “and” or “or”. For example, “A and/or B” means “A, B, or both A and B” and “A, B, C, and/or D” means “A, B, C, D, or a combination thereof” and said “A, B, C, D, or a combination thereof” means any subset of A, B, C, and D, for example, a single member subset (e.g., A or B or C or D), a two-member subset (e.g., A and B; A and C; etc.), or a three-member subset (e.g., A, B, and C; or A, B, and D; etc.), or all four members (e.g., A, B, C, and D).

As used herein, the phrase “one or more of”, e.g., “one or more of A, B, and/or C” means “one or more of A”, “one or more of B”, “one or more of C”, “one or more of A and one or more of B”, “one or more of B and one or more of C”, “one or more of A and one or more of C” and “one or more of A, one or more of B, and one or more of C”.

The phrase “comprises or consists of A” is used as a tool to avoid excess page and translation fees and means that in some embodiments the given thing at issue: comprises A or consists of A. For example, the sentence “In some embodiments, the composition comprises or consists of A” is to be interpreted as if written as the following two separate sentences: “In some embodiments, the composition comprises A. In some embodiments, the composition consists of A.”

Similarly, a sentence reciting a string of alternates is to be interpreted as if a string of sentences were provided such that each given alternate was provided in a sentence by itself. For example, the sentence “In some embodiments, the composition comprises A, B, or C” is to be interpreted as if written as the following three separate sentences: “In some embodiments, the composition comprises A. In some embodiments, the composition comprises B. In some embodiments, the composition comprises C.” As another example, the sentence “In some embodiments, the composition comprises at least A, B, or C” is to be interpreted as if written as the following three separate sentences: “In some embodiments, the composition comprises at least A. In some embodiments, the composition comprises at least B. In some embodiments, the composition comprises at least C.”

To the extent necessary to understand or complete the disclosure of the present invention, all publications, patents, and patent applications mentioned herein are expressly incorporated by reference therein to the same extent as though each were individually so incorporated.

Having thus described exemplary embodiments of the present invention, it should be noted by those skilled in the art that the within disclosures are exemplary only and that various other alternatives, adaptations, and modifications may be made within the scope of the present invention. Accordingly, the present invention is not limited to the specific embodiments as illustrated herein, but is only limited by the following claims.

1. A nucleic acid molecule which comprises or consists of a sequence having 100% sequence identity to at least 17, at least 18, or at least 19 nucleotides of a sequence selected from the group consisting of SEQ ID NOs: 1-43 and 87-118, preferably selected from the group consisting of SEQ ID NOs: 1-41 and 87-118.

2. A guide RNA comprising the nucleic acid molecule according to claim 1.

3. A guide RNA comprising a guide sequence having 100% sequence identity to at least 17, at least 18, or at least 19 nucleotides of a sequence selected from the group consisting of SEQ ID NOs: 1-41.

4. A nucleic acid molecule comprising a sequence that encodes the guide RNA according to claim 2.

5. A composition comprising one or more nucleic acid molecules according to claim 1 or one or more guide RNAs comprising a nucleic acid molecule according to claim 1.

6. A kit comprising one or more nucleic acid molecules according to claim 1 or one or more guide RNAs comprising a nucleic acid molecule according to claim 1, and/or a composition thereof.

7. A method of modifying a dystrophin gene in a cell or a subject, which comprises introducing into the cell or subject (a) a Cas protein or a nucleotide sequence encoding the Cas protein; and (b1) a single guide RNA according to claim 2 or encoding nucleotide molecule(s) thereof, or (b2) a first gRNA and a second gRNA or encoding nucleotide molecule(s) thereof, wherein the first gRNA and/or the second gRNA is a guide RNA according to claim 2.

8. The method according to claim 7, wherein the Cas protein is a class 2 CRISPR/Cas endonuclease.

9. The method according to claim 8, wherein the Cas protein is a type II CRISPR/Cas endonuclease.

10. The method according to claim 9, wherein the Cas protein is a Cas9 endonuclease.

11. The method according to claim 7, wherein the cell is a muscle cell, a pericyte, an induced pluripotent stem (iPS) cell, or a stem cell.

12. A method of treating Duchenne muscular dystrophy or Becker muscular dystrophy in a subject, which comprises modifying the dystrophin gene in the subject according to the method according to claim 7.

13. The method according to claim 7, wherein the cell is a human cell and/or the subject is human.

14. The method according to claim 7, wherein the cell is genetically modified to have the dystrophin gene and/or the subject is an animal genetically modified to have the dystrophin gene.
